(19) United States
(12) Patent Application Publication
(10) Pub. No.: US 2025/0367099 A1
(43) Pub. Date: Dec. 4, 2025

(54) UROLITHIN COMBINATIONS

(71) Applicants: Davide D'Amico, Renens (CH); Julie Faitg, Lausanne C (CH); Christopher L. Rinsch, Lausanne (CH); Anurag Singh, Lausanne (CH)

(72) Inventors: Davide D'Amico, Renens (CH); Julie Faitg, Lausanne C (CH); Christopher L. Rinsch, Lausanne (CH); Anurag Singh, Lausanne (CH)

(21) Appl. No.: 19/227,214

(22) Filed: Jun. 3, 2025

(30) Foreign Application Priority Data
Jun. 3, 2024 (GB) 2407882.6
Jun. 10, 2024 (GB) 2408217.4

(51) Int. Cl. A61K 8/49; A61K 8/02; A61K 8/11; A61K 8/27; A61K 8/31; A61K 8/35; A61K 8/68; A61Q 19/08

(52) U.S. Cl. CPC A61K 8/498; A61K 8/0216; A61K 8/11; A61K 8/27; A61K 8/31; A61K 8/355; A61K 8/68; A61Q 19/08

(57) ABSTRACT

Described is a composition comprising:
(a) a compound of formula (I), or a salt, prodrug, metabolite or derivative thereof;

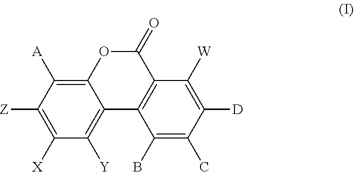

wherein:
A, B, C, D, W, X, Y and Z are each independently selected from H and OH; and
(b) one of more agents selected from agents for improving skin health, wherein the agents for improving skin health are selected from one or more of the following:
   (i) one or more ceramides, for example, one or more phytoceramides;
   (ii) a carotenoid, for example, lycopene and/or lutein;
   c. zinc or a salt thereof;
   d. astaxanthin; and optionally
   e. L-lysine;
   f. one or more NAD boosters, for example, nicotinamide and/or co-enzyme Q₁₀;
   g. eggshell membrane or an extract thereof; and/or
   h. biotin, or a derivative thereof.

### RELATED APPLICATIONS

This application claims the benefit of priority to UK Patent Application Nos. 2408217.4, filed Jun. 10, 2024; and 2407882.6, filed Jun. 3, 2024.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA).

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, α1-antitrypsin deficiency, ischemia/reperfusion injury, inflammation, aging of the skin, inflammatory bowel disease, Crohn's disease, obesity, metabolic syndrome, type II diabetes mellitus, hyperlipidemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

### SUMMARY

The invention relates to compositions comprising urolithins with active agents, particularly compositions comprising urolithins and agents which promote skin health. The invention also relates to such compositions, for use for the treatment or prevention of diseases, disorders and conditions, and for use in non-therapeutic treatments.

As discussed above, urolithins, for example, urolithin A, are effective in a variety of conditions, and for improving health, in individuals of all ages, particularly for promoting healthy aging and increasing healthspan. In the development of nutritional supplements that target a specific health benefit, we have identified a number of combinations of urolithins with other active ingredients, which can surprisingly increase the benefit of urolithin administration. In particular, urolithin has unexpectedly been found to work synergistically with other active ingredients, for example, combining a urolithin with zinc shows synergistic effects in reducing inflammation, and combining a urolithin with astaxanthin shows synergistic effects in increasing mitochondrial respiration.

Therefore, according to the present invention there is provided a composition comprising:
(a) a compound of formula (I), or a salt, prodrug, metabolite or derivative thereof;

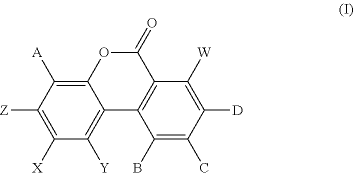

wherein:
A, B, C, D, W, X, Y and Z are each independently selected from H and OH; and
(b) one or more agents selected from agents for improving skin health.

The compositions of the invention are surprisingly effective and efficient for the provision of benefits to a subject taking the composition. The invention is described below in further detail.

### BRIEF DESCRIPTION OF THE FIGURE

FIG. 1A show the treatment groups of the study in Example 2.

FIG. 1B also show the treatment groups of the study in Example 2.

FIG. 2 shows the assignment of treatment groups and test areas in Example 2.

### DETAILED DESCRIPTION

According to a first aspect of the present invention, there is provided a composition comprising:
(a) a compound of formula (I), or a salt, prodrug, metabolite or derivative thereof;

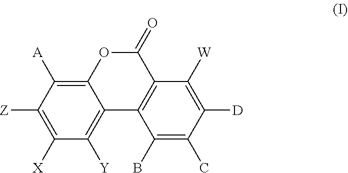

wherein:
A, B, C, D, W, X, Y and Z are each independently selected from H and OH; and
(b) one or more agents selected from agents for improving skin health, wherein the agents for improving skin health are selected from one or more of the following:
   (i) one or more ceramides, for example, one or more phytoceramides;
   (ii) a carotenoid, for example, lycopene and/or lutein;
   (iii) zinc or a salt thereof;
   (iv) L-lysine;
   (v) one or more nicotinamide adenine dinucleotide (hereinafter referred to as NAD) boosters, for example, nicotinamide and/or co-enzyme Q₁₀;
   (vi) eggshell membrane or an extract thereof;
   (vii) astaxanthin; and/or
   (viii) biotin, or a derivative thereof.

A composition comprising:
(a) a compound of formula (I), or a salt, prodrug, metabolite or derivative thereof;

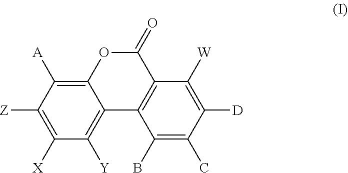

wherein:
A, B, C, D, W, X, Y and Z are each independently selected from H and OH; and
(b) one of more agents selected from agents for improving skin health, wherein the agents for improving skin health are selected from one or more of the following:
   (i) one or more ceramides, for example, one or more phytoceramides;
   (ii) a carotenoid, for example, lycopene and/or lutein;
   (i) zinc or a salt thereof;
   (ii) astaxanthin; and optionally
   (iii) L-lysine;
   (iv) one or more NAD boosters, for example, nicotinamide and/or co-enzyme Q₁₀;
   (v) eggshell membrane or an extract thereof; and/or
   (vi) biotin, or a derivative thereof.

Preferably, the composition according to the present invention comprises:
(a) a compound of formula (I), or a salt, prodrug, metabolite or derivative thereof, as defined above; and
(b) one or more ceramides, for example, one or more phytoceramides.

More preferably, the composition according to the present invention further comprises one or more carotenoids (for example, lycopene or lutein).

Even more preferably, the composition according to the present invention further comprises a zinc salt, for example, a zinc salt selected from zinc oxide, zinc sulphate, zinc acetate, zinc citrate, zinc gluconate, zinc picolinate, zinc orotate, and zinc bisglycinate.

Preferably, the composition according to the present invention comprises
(a) a compound of formula (I), or a salt, prodrug, metabolite or derivative thereof;
(b) one or more phytoceramides;
(c) a carotenoid, for example, lycopene and/or lutein; and
(d) a zinc salt, for example, a zinc salt selected from zinc oxide, zinc sulphate, zinc acetate, zinc citrate, zinc gluconate, zinc picolinate, zinc orotate, and zinc bisglycinate.

Preferably, the carotenoid is lycopene.

According to a composition of the present invention there is provided:
(a) a compound of formula (I), or a salt, prodrug, metabolite or derivative thereof;

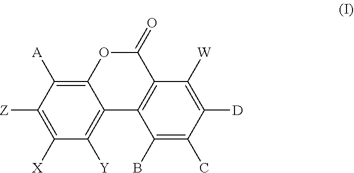

wherein:
A, B, C, D, W, X, Y and Z are each independently selected from H and OH; and
(b) one of more agents selected from agents for improving skin health, wherein the agents for improving skin health are selected from one or more of the following:
   (i) one or more ceramides, for example, one or more phytoceramides;
   (ii) a carotenoid, for example, lycopene and/or lutein;
   (iii) zinc or a salt thereof;
   (iv) astaxanthin; and optionally
   (v) L-lysine;
   (vi) one or more NAD boosters, for example, nicotinamide and/or co-enzyme Q₁₀;
   (vii) eggshell membrane or an extract thereof; and/or
   (vii) biotin, or a derivative thereof.

According to a further embodiment of the present invention, there is provided a composition, as described above, comprising one or more NAD boosters, for example, nicotinamide and/or co-enzyme Q₁₀.

Another embodiment provides a composition according to the present invention further comprising one or more agents selected from:
(i) egg shell membrane or an extract thereof;
(ii) collagen;
(iii) L-lysine;
(iv) nicotinamide and/or reservatrol;
(v) astaxanthin; and
(vi) Miliacin encapsulated by polar lipid.

Preferably, the composition, according to the present invention comprises:
(a) a compound of formula (I), or a salt, prodrug, metabolite or derivative thereof, as defined above; and
(b) biotin or a derivative thereof.

More preferably, the biotin or a derivative thereof is selected from free biotin, biocytin (e-biotin-L-Lysine), L-biotin sulphoxide, and D-biotin sulphoxide. Even more preferably, the biotin derivatives comprise biotin esters, biotinamides, carboxybiotin, carboxybiotin esters and carboxybiotinamides, including pharmaceutically-acceptable salts thereof.

According to a preferred composition of the present invention, the compound of formula (I) is selected from urolithin A, urolithin B, urolithin C and urolithin D, for example, urolithin A.

According to a second aspect of the present invention, there is provided a composition according to the present invention for use in a method of prevention and/or treatment of a skin condition, disease or disorder.

Preferably, the composition of the present invention is for use in the treatment of the skin condition, disease or disorder is a skin condition, disease or disorder associated with inadequate mitochondrial activity.

According to a preferred aspect of the present invention, there is provided the use of a composition according to the present invention for maintaining or enhancing skin health.

According to yet another aspect of the present invention, there is provided a composition according to the present invention for use in:
a. improving skin health;
b. for maintaining or enhancing skin energy;
C. for maintaining or enhancing skin collagen;
d. reducing skin biological aging;
e. supporting healthy skin aging;
f. reducing skin wrinkles, and/or fine lines;
g. improving skin mitochondrial function;
h. increasing skin collagen levels;
i. improving skin hydration;
j. promoting replenishment of skin ceramide levels;
k. improving skin elasticity and barrier function;
I. protecting against free radical damage;
m. improving skin dryness;
n. improving photodamaged skin, and/or
o. Improving skin health in skin prone to redness, inflammation, acne or dryness.

The compositions of the present invention described herein may be provided as a soft gel (for example in a capsule having a shell), as a gummy, as a powder as a tablet, as a lotion or as a cream.

A preferred embodiment of the invention is a composition as described herein for use in combination with a topical skin formulation comprising urolithin A to prevent or improve skin aging. Preferably, the topical skin formulation is a cosmetic topical cream comprising about 0.5% to about 5% of urolithin A. More preferably, it comprises about 1% of urolithin A.

Preferably, the composition of the invention is an oral composition to be administered with a topical skin formulation also comprising urolithin A.

An additional embodiment of the invention is a cosmetic composition comprising a composition as described herein.

According to another aspect of the present invention, there is provided a kit comprising:
(a) a composition according to the present invention, as described above; and
(b) a container, or containers, for containing said composition; and
(a) optionally instructions for simultaneous, separate or sequential administration with a topical skin formulation, comprising a compound of formula (I), for example, urolithin A.

Preferably, the kit comprises:
(b) a composition according to the present invention for oral administration;
(c) a topical skin formulation, comprising a compound of formula (I), for example, urolithin A;
(d) a container, or containers, for containing said compositions; and
(e) optionally instructions for simultaneous, separate or sequential administration.

### Compositions Comprising an Agent for Improving Skin Health

The invention provides a composition comprising:
(a) a compound of formula (I), or a salt, prodrug, metabolite or derivative thereof; and
(b) one or more agents selected from agents for improving skin health.

An agent for improving skin health may be selected from:
(i) one or more skin barrier protectants (for example a ceramide, for example, one or more phytoceramides);
(ii) one or more skin elasticity enhancers (for example a carotenoid, for example, lycopene and/or lutein); and
(iii) one or more agent to prevent photodamage (for example zinc).

An agent for improving skin health may be selected from:
(i) one or more ceramides, for example, one or more phytoceramides;
(ii) a carotenoid, for example, lycopene and/or lutein; and
(iii) zinc.

Therefore, in a further embodiment, the invention provides a composition comprising:
(a) a mitophagy activator, for example, urolithin A;
(b) one or more skin barrier protectants (for example a ceramide, for example, one or more phytoceramides);
(c) one or more skin elasticity enhancers (for example a carotenoid, for example, lycopene and/or lutein); and
(d) one or more agent to prevent photodamage (for example zinc).
(e) One or more antioxidants (for example astaxanthin)

In an embodiment of the invention, there is provided a composition comprising:
(a) a compound of formula (I), or a salt, prodrug, metabolite or derivative thereof; and
(b) one or more ceramides (for example, one or more phytoceramides).

The ceramides may comprise an extract comprising a mixture of ceramides from a natural source. The ceramide may, for example, comprise ceramide 1, ceramide 3 and/or ceramide 6-II. Certain ceramides may be obtained from dairy products or eggs. The ceramide may be a phytoceramide, i.e. of plant origin. For example, it may be from plant oil origin, for example from jojoba oil, safflower oil or from grapeseed oil. Certain phytoceramides can be obtained from soybeans, rice, oats, peas, potatoes, sweet potato, sesame, coconut, grape seeds, peanuts, millet, spinach, wheat germ and some fruits, for example, peach. In one embodiment, the phytoceramides are derived from rice, for example, CeralOK® (Anderson advanced ingredients, Irvine, California, USA). The ceramides or phytoceramides, in composition of the inventions, can be a mixture of phytoceramides, for example, including β-sitosterol-3-O-glucoside.

In an embodiment of the invention, there is provided a composition comprising:
(a). a compound of formula (I), or a salt, prodrug, metabolite or derivative thereof;
(b). a carotenoid.

The carotenoid may, for example, be selected from lycopene and lutein.

In an embodiment of the invention, there is provided a composition comprising:
(a). a compound of formula (I), or a salt, prodrug, metabolite or derivative thereof;
(b). zinc.

The zinc is typically provided in the form of a salt, for example as zinc oxide, zinc sulphate, zinc acetate, zinc glycerate, and zinc monomethionine, zinc citrate, zinc gluconate, zinc picolinate, zinc orotate, and zinc bisglycinate. In a further embodiment, the zinc salt is selected from one or more salts selected from zinc picolinate, zinc citrate, zinc gluconate, zinc bisglycinate, zinc monomethionine and zinc acetate. In one embodiment, the zinc salt is zinc picolinate. The zinc has been found to have has a synergistic effect with a compound of formula (I) and in particular with urolithin A.

In an embodiment of the invention, there is provided a composition comprising:
(a) a compound of formula (I), or a salt, prodrug, metabolite or derivative thereof;
(b) astaxanthin.

The astaxanthin is an antioxidant and has been found to have a synergistic effect with a compound of formula (I) and in particular with urolithin A.

In an embodiment of the invention, there is provided a composition comprising:
(a). a compound of formula (I), or a salt, prodrug, metabolite or derivative thereof;
(b). one or more ceramides, for example, one or more phytoceramides; and
(c). a carotenoid, for example, lycopene and/or lutein.

In an embodiment of the invention, there is provided a composition comprising:
(a). a compound of formula (I), or a salt, prodrug, metabolite or derivative thereof;
(b). one or more ceramides, for example, one or more phytoceramides; and
(c). zinc.

In an embodiment of the invention, there is provided a composition comprising:
(a). a compound of formula (I), or a salt, prodrug, metabolite or derivative thereof;
(b). a carotenoid, for example, lycopene and/or lutein; and
(c). zinc.

In an embodiment of the invention, there is provided a composition comprising:
(a). a compound of formula (I), or a salt, prodrug, metabolite or derivative thereof;
(b). one or more ceramides, for example, one or more phytoceramides;
(c). a carotenoid, for example, lycopene and/or lutein; and
(d). zinc.

In a further embodiment of the invention, the composition also optionally includes an NAD booster (for example nicotinamide and/or co-enzyme Q₁₀). Accordingly, there is provided a composition comprising:
(a) a compound of formula (I), or a salt, prodrug, metabolite or derivative thereof; and/or
(b) one or more ceramides, for example, one or more phytoceramides; and/or
(c) one or more carotenoids, for example, lycopene or lutein; and/or
(d) zinc;
(e) astaxanthin; and
(f) one or more NAD boosters, for example, nicotinamide and/or co-enzyme Q₁₀.

NAD boosters include niacin, niacinamide, nicotinamide, nicotinamide riboside (NR), nicotinamide mononucleotide (NMN), tryptophan, trigonelline and pyrroloquinoline quinone. A number of variants of co-enzyme Q₁₀ are known; including ubiquinol and ubiquinone. Such variants are included herein within the term co-enzyme Q₁₀ unless stated otherwise. For example, a composition of the invention includes ubiquinol. In an alternative embodiment a composition of the invention includes ubiquinone.

In a further embodiment of the invention, the composition also optionally includes L-lysine. Accordingly, there is provided a composition comprising:
(a) a compound of formula (I), or a salt, prodrug, metabolite or derivative thereof; and/or
(b) one or more ceramides, for example, one or more phytoceramides; and/or
(c) a carotenoid, for example, lycopene and/or lutein; and/or
(d) zinc; and/or L-lysine; and/or astaxanthin
(e) Optionally one or more NAD boosters, for example, nicotinamide and/or co-enzyme Q₁₀.

In a further embodiment of the invention, there is provides a composition comprising:
(a) a compound of formula (I), or a salt, prodrug, metabolite or derivative thereof;
(b) one or more ceramides, for example, one or more phytoceramides; and
(c) one of more agents selected from:
   (i) eggshell membrane or an extract thereof, for example, Ovomet®;
   (ii) collagen;
   (iii) L-lysine

In a further embodiment of the invention, there is provided a composition comprising:
(a) a compound of formula (I), or a salt, prodrug, metabolite or derivative thereof;
(b) one or more ceramides, for example, one or more phytoceramides;
(c) one of more agents selected from:
   (i) egg shell membrane or an extract thereof;
   (ii) collagen; and
   (iii) L-lysine; and
(d) one of more agents selected from
   (i) nicotinamide and/or resveratrol;
   (ii) astaxanthin; and
   (iii) biotin; and
   (iv) Miliacin encapsulated by polar lipid.

Egg shell membrane or an extract thereof is available, for example from Eggnovo (https://eggnovo.com/) under the tradename Ovomet®. Miliacin encapsulated by polar lipid is available, for example, from Robertet (https://www.robertet.com/en/), under the tradename Keranut®.

In a further embodiment of the invention, there is provided a composition comprising:
(a) a compound of formula (I), or a salt, prodrug, metabolite or derivative thereof; and
(b) a sirtuin-activating compound, for example, fisetin.

In one embodiment, fisetin is administered at a dose of about 0.5 mg to about 2500 mg (e.g., about 0.5 mg to about 20 mg, about 1 mg to about 10 mg, about 2 mg to about 6 mg, about 25 mg to about 500 mg, about 50 mg to about 300 mg, about 100 mg to about 200 mg, about 200 mg to about 1500 mg, about 300 mg to about 1200 mg, or about 400 mg to about 750 mg). Fisetin can be administered once a day, twice a day, or three or more times a day. For example, fisetin can be administered at a dose of about 0.5 mg to about 2000 mg (e.g. any of the subranges or doses within this range disclosed herein) twice a day. Fisetin can be administered at an amount of about 1 mg to about 5000 mg daily (e.g., about 1 mg to about 40 mg, about 2 mg to about 20 mg, about 4 mg to about 12 mg, about 50 mg to about 1000 mg, about 100 mg to about 800 mg, about 150 mg to about 500 mg, about 200 mg to about 400 mg, about 500 mg to about 2000 mg, about 700 mg to about 1800 mg, or about 800 mg to about 1500 mg daily). In some embodiments, fisetin is administered at an amount of about 8 mg daily (e.g., about 4 mg twice a day).

In some embodiments, fisetin is administered at an amount of about 0.05 to about 60 mg/kg of the subject's body weight (e.g., about 0.05 to about 0.15, about 0.4 to about 12, about 30 0.6 to about 10, about 0.8 to about 8, about 1 to about 6, about 1.5 to about 26, about 2 to about 24, about 2.5 to about 22, or about 3 to about 20 mg/kg of the subject's body weight).

In a further embodiment of the invention, there is provided a composition comprising:
(a) a compound of formula (I), or a salt, prodrug, metabolite or derivative thereof; for example, urolithin A, or a salt, prodrug, metabolite or derivative thereof;
(b) one or more ceramides, for example, one or more phytoceramides;
(c) lycopene; and
(d) a salt of zinc;
(e) astaxanthin; and
(f) optionally nicotinamide;
(g) optionally lysine;
(h) optionally co-enzyme Q₁₀;
(i) optionally lutein; and
(j) optionally orthosilicic acid.

In further embodiment of the invention, there is provided a composition comprising:
(a) a compound of formula (I), or a salt, prodrug, metabolite or derivative thereof; for example, urolithin A, or a salt, prodrug, metabolite or derivative thereof;
(b) one or more ceramides, for example, one or more phytoceramides;
(c) lutein;
(d) zinc;
(e) astaxanthin; and
(f) optionally nicotinamide;
(g) optionally lysine;
(h) optionally co-enzyme Q₁₀; and
(i) optionally orthosilicic acid.

Preferably, the composition of the present invention comprising about 125 mg to about 600 mg of the compound of formula (I), about 1 mg to about 400 mg of phytoceramides, about 2 mg to about 200 mg of lutein, about 1 mg to about 11 mg zinc and about 1 to about 30 mg astaxanthin.

More preferably, the compound of formula (I) is in the range of about 130 mg to about 550 mg, about 150 mg to about 500 mg, about 200 mg to about 450 mg, about 250 mg to about 400 mg, about 300 mg to about 350 mg, for example about 125 mg, about 130 mg, about 140 mg, about 145 mg, about 150 mg, about 200 mg, about 250 mg, about 300 mg and 350 mg by weight.

Preferably, the phytoceramides are in the range of about 10 mg to about 350 mg, for example about 20 mg to about 300 mg, about 30 mg to about 250 mg, about 40 mg to about 200 mg, about 50 mg to about 150 mg, for example about 10 mg, about 20 mg, about 30 mg, about 40 mg, about 50 mg, about 100 mg, about 150 mg, about 200 mg, about 250 mg, about 300 mg and about 350 mg.

Preferably, lutein is in the range of about 2 mg to about 200 mg, for example about 4 mg to about 150 mg, about 6 mg to about 140 mg, about 8 mg to about 130 mg, about 10 mg to about 120 mg, about 12 mg to about 120 mg, about 14 mg to about 110 mg, about 14 mg to about 100 mg, about 16 mg to about 90 mg, about 18 mg to about 80 mg, about 20 mg to about 70 mg, about 30 mg to about 60 mg, about 30 mg to about 50 mg, for example about 2 mg, about 3 mg, about 4 mg, about 5 mg, about 6 mg, about 7 mg, about 8 mg, about 9 mg, about 10 mg or about 12 mg.

Preferably, zinc is in the range of about 1 mg to about 30 mg, for example about 2 mg to about 28 mg, about 3 mg to about 26 mg, about 4 mg to about 24 mg, about 5 mg to about 22 mg, about 6 mg to about 20 mg, about 7 mg to about 18 mg, about 8 mg to about 16 mg, about 9 mg to about 14 mg, about 10 mg to about 12 mg, for example about 10 mg, about 11 mg, about 8 mg, about 6 mg, about 4 mg and about 2 mg.

Preferably the astaxanthin is in the range of about 1 mg to 30 mg, for example about 2 mg to about 28 mg, about 3 mg to about 26 mg, about 4 mg to about 24 mg, about 5 mg to about 22 mg, about 6 mg to about 20 mg, about 7 mg to about 18 mg, about 8 mg to about 16 mg, about 9 mg to about 14 mg, about 10 mg to about 12 mg, for example about 10 mg, about 11 mg, about 8 mg, about 6 mg, about 4 mg and about 2 mg.

The agent for improving skin health can also be biotin (vitamin B7), or a derivative thereof. Therefore, in a further embodiment, there is provided a composition comprising:
(a) a compound of formula (I), or a salt, prodrug, metabolite or derivative thereof; for example, urolithin A, or a salt, prodrug, metabolite or derivative thereof; and
(b) biotin, or a derivative thereof.

In a further embodiment of the invention there is provided composition, comprising:
(a) a compound of formula (I), wherein the compound of formula (I) is a urolithin, for example, urolithin A, urolithin B, urolithin C or urolithin D, such as urolithin A; and;
(b) biotin, or a derivative thereof.

In a further embodiment of the invention there is provided a composition, comprising:
(a) about 100 mg to about 2000 mg of a compound of formula (I), or a salt thereof, for example, urolithin A; and
(b) about 10 μg to about 50 mg of biotin, or a derivative thereof.

In one embodiment, the biotin, or derivative thereof, is selected from free biotin, biocytin (e-biotin-L-Lysine), L-biotin sulphoxide, and D-biotin sulphoxide.

In a further embodiment, the biotin derivatives comprises biotin esters, biotinamides, carboxybiotin, carboxybiotin esters and carboxybiotinamides, including pharmaceutically-acceptable salts thereof.

Composition of the invention are also useful for improving nail health and/or improving hair health. Therefore, in a further embodiment of the invention there is provided a composition comprising:
(a) a compound of formula (I), or a salt, prodrug, metabolite or derivative thereof;

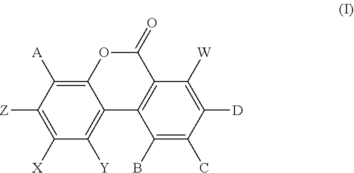

wherein:
A, B, C, D, W, X, Y and Z are each independently selected from H and OH; and
(b) one of more agents selected from agents for improving hair health, wherein the agent for improving hair health is selected from one of more of the agents listed above for improving skin health.

In a further embodiment of the invention there is provided a composition comprising:
(a) a compound of formula (I), or a salt, prodrug, metabolite or derivative thereof;

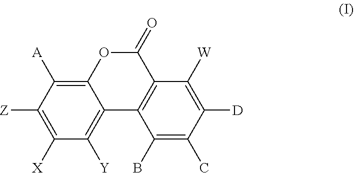

wherein:
A, B, C, D, W, X, Y and Z are each independently selected from H and OH; and
(b) one of more agents selected from agents for improving nail health, wherein the agent for improving nail health is selected from one of more of the agents listed above for improving skin health.

Typically, a composition of the invention comprises from 125 mg to 2500 mg of the compound of formula (I). As described in further detail below, a preferred compound of formula (I) is Urolithin A. That is to say that a composition of the invention may comprise from 125 mg to 2500 mg of Urolithin A. More preferably, a composition of the invention may comprise 250 mg to 2500 mg of Urolithin and even more preferably, 500 mg of Urolithin.

In a further embodiment, a composition of the invention comprises about 0.1 mg to 10 mg ceramides, for example, about 0.5 mg to about 5 mg ceramides, for example about 0.7 mg to about 3 mg ceramides, for example, about 0.8 mg ceramides to about 2 mg ceramides, for example, about 1 mg to about 1.5 mg ceramides. In a further embodiment the ceramides are phytoceramides.

In a further embodiment, the ceramides of the invention are formulated with a carrier, for example, cyclodextrin. Composition of the invention comprises about 1 mg to about 100 mg of the formulated ceramides, for example, about 10 milligrams to about 100 milligrams of the formulated ceramides or about 1 mg to about 50 mg of the formulated ceramides, such as of the formulated phytoceramides, for example about 20 milligrams to about 80 milligrams of the formulated ceramides, for example about 20 milligrams to about 40 milligrams of the formulated ceramides, for example 40 milligrams of the formulated ceramides. The formulated ceramide may be phytoceramides. A composition of the invention may comprise about 1 to about 50 mg of formulated phytoceramides. More preferably, a composition comprises about 20 mg or about 40 mg of formulated phytoceramides. Preferably, a composition comprises about 20 mg of formulated phytoceramides. In another embodiment, a composition of the invention comprises about 40 mg of formulated phytoceramides. In one embodiment, the formulated ceramide contains about 0.5% to about 10% (w/w) of ceramides, such as phytoceramides, such as about 1% to about 5% (w/w) ceramides, for example, about 2% to about 4% ceramides. In a further embodiment, the formulated ceramides comprise β-Sitosterol-3-O-Glucosides, for example about 0.05% to about 1% (w/w) β-Sitosterol-3-O-Glucosides, such as about 0.1% to about 0.5% (w/w) β-Sitosterol-3-O-Glucosides, for example, about 0.1% to about 0.4% β-Sitosterol-3-O-Glucosides.

In a further embodiment, the ceramides, for example, phytoceramides further comprise one of more of the following:
(a) diglycerides and/or triglcerides;
(b) phospholipids, for example, phosphatidylethanolamine, phosphatidylcholine and/or phosphatidylinositol; and/or
(c) glycolipids, for example digalactosyl-diglycerides.

In further embodiments, the ceramides, for example, phytoceramides are provided in an extract, for example, a wheat seed extract, for example, Lipowheat® (Robertet, Grass, France). Such extract can be provided as an oil or a powder. Such extracts comprise a mixture of components, for example:
(i) about 35% to about 40% (w/w) diglycerides and/or triglycerides;
(ii) about 7% to about 10% phosholipids, of which, for example, the phospholipids comprise:
   a. about 25% to about 30% (w/w) phosphatidylethanolamine,
   b. about 30% to about 35% (w/w) phosphatidylcholine, and/or
   c. about 30% to about 35% (w/w) phosphatidylinositol;
(iii) about 20% to about 30% (w/w) glycolipids such as digalactosyl-diglycerides;
(iv) about 2% to about 4% ceramides and glycoceramides, of which, for example, comprise
   a. about 0.3% to about 0.6% (w/w) ceramides; and/or
   b. about 1.5% to about 3% glycocermides.
Or for example:
(a) less than or equal to about 1% (w/w) diglycerides and/or triglycerides;
(b) about 25% to about 30% phosholipids, of which, for example, the phospholipids comprise:
   a. about 25% to about 30% (w/w) phosphatidylethanolamine,
   b. about 30% to about 35% (w/w) phosphatidylcholine, and/or
   c. about 30% to about 35% (w/w) phosphatidylinositol;
(c) about 50% to about 55% (w/w) glycolipids such as digalactosyl-diglycerides;
(d) about 3% to about 5% ceramides and glycoceramides, of which, for example, comprise
   a. about 0.3% to about 0.6% (w/w) ceramides; and/or
   b. about 2% to about 4% glycocermides.

Ceramides are compounds composed of sphingosine and a fatty acid joined by an amide bond. They are compounds of the general structure:

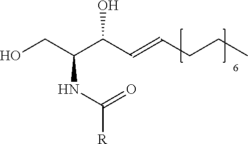

in which R is the alkyl portion of a fatty acid. Ceramides comprise ceramides and/or glycosylceramides.

In one embodiment, ceramides comprise glucosyl ceramides. In a further embodiment, ceramides comprise β-sitosterol-3-O-Glucosides.

A ceramide for use in the invention may include the products available under the names Ceramide NP, Ceramide NS, Ceramide NG, and/or CeralOK® (Anderson Advance Ingredients, Irvine, CA, USA).

In a further embodiment, a composition of the invention comprises about 10 milligrams to about 100 milligrams carotenoids, for example about 20 milligrams to about 100 milligrams, about 20 milligrams to about 80 milligrams carotenoids, for example about 20 milligrams to about 40 milligrams carotenoids, for example 40 milligrams carotenoids or about 20 milligrams. A composition of the invention may comprise about 1 milligram to about 50 milligrams, for example about 5 milligrams. As mentioned above, the carotenoid can, for example, be lycopene or lutein. The lutein in the composition may be formulated lutein, for example Lutamax 2020 Free. Preferably, where the carotenoid is lycopene, the composition comprises about 20 milligrams or about 40 milligrams or lycopene. Where the carotenoid is formulated lutein, the composition preferably comprises about 5 milligrams, about 10 milligrams, about 20 milligrams, about 30 milligrams or about 40 milligrams of lutein.

In a further embodiment, a composition of the invention comprises about 2 milligrams to about 50 milligrams elemental zinc, for example about 2.5 mg to about 50 mg, 5 mg to about 50 mg, about 10 mg to about 40 mg zinc, about 30 mg to about 50 mg, about 5 milligrams to about 20 milligrams zinc, about 5 mg to about 10 mg, for example about 2.5 mg, about 2.5 mg, 10 milligrams zinc, about 15 mg zinc, about 25 mg zinc, about 50 mg zinc. Zinc is included in the form of a salt for example, zinc picolinate, zinc citrate, zinc acetate, zinc glycerate, zinc sulphate, Zinc bisglycinate, and zinc monomethionine. Preferably, the zinc bysglycinate (27% zinc) is used.

In a further embodiment, if co-enzyme Q₁₀ is present, then a composition of the invention may comprise about 25 milligrams to about 300 milligrams co-enzyme Q₁₀, for example about 50 milligrams to about 200 milligrams co-enzyme Q₁₀, for example about 150 milligrams co-enzyme Q₁₀. If nicotinamide is present, then a composition of the invention may comprise about 250 milligrams to about 500 milligrams nicotinamide.

In a further embodiment, if lysine is present, then a composition of the invention may comprise about 200 milligrams to about 2 grams L-lysine, for example about 500 milligrams to about 2 grams, for example about 500 milligrams to about 1500 milligrams, for example about 750 milligrams to about 1250 milligrams. For example, a composition of the invention may comprise about 1 gram to about 2 grams L-lysine.

An agent for improving skin health included in compositions of the invention may also be selected from biotin. Accordingly, in an embodiment of the invention, there is provided a composition of the invention, further comprising biotin:

In a further embodiment, if biotin is present, then a composition of the invention may comprise about 20 micrograms to 10000 micrograms biotin, for example about 50 micrograms to 5000 micrograms biotin, for example about 200 micrograms to 2000 micrograms biotin, for example about 2500 micrograms to 5000 micrograms biotin.

In a further embodiment, if eggshell membrane is present then a composition of the invention may comprise about 300 milligrams to about 400 milligrams eggshell membrane.

In a further embodiment, if collagen or collagen peptides are present, then a composition of the invention may comprise about 10 milligrams to about 10 grams collagen or collagen peptides, for example about 100 milligrams to about 5 grams collagen or collagen peptides, for example about 200 milligrams to about 2 grams collagen or collagen peptides.

In a further embodiment, if resveratrol is present, then a composition of the invention may comprise about 1 milligrams to about 1500 milligrams resveratrol, for example, 50 milligrams to about 1500 milligrams resveratrol for example about 100 milligrams to about 1000 milligrams resveratrol, for example about 250 milligrams to about 500 milligrams resveratrol. Resveratrol may also be in the form of a glycosylated derivative, for example, piceid, piceatannol glucoside, and resveratroloside. Resveratrol also includes metabolites of resveratrol, for example, piceatannol.

In a further embodiment, the composition may comprise the resveratrol analogue, pterostilbene.

In a further embodiment, if astaxanthin is present, then a composition of the invention may comprise about 2 milligrams to about 50 milligrams astaxanthin, for example about 4 milligrams to about 40 milligrams astaxanthin, for example about 4 milligrams to about 20 milligrams astaxanthin, for example about 4 milligrams to about 8 milligrams astaxanthin, for example about 6 milligrams to about 8 milligrams for example, about 4 mg to about 18 mg, for example about 5 mg to about 20 mg, for example, about 6 mg, about 8 mg, about 12 mg, about 16 mg, about 18 mg, about 20 mg. Preferably, the composition comprises astaxanthin at about 6 mg or 8 mg.

A preferred embodiment of the invention provides a composition comprising Urolithin A, Phytoceramides, lycopene, zinc, co-enzyme Q₁₀ and further inactive carriers.

Another preferred embodiment of the invention provides a composition comprising Urolithin A and phytoceramides and further inactive carriers.

Another preferred embodiment of the invention provides a composition comprising Urolithin A and lycopene and further inactive carriers.

Another preferred embodiment of the invention provides a composition comprising Urolithin A and zinc in salt form and further inactive carriers.

A preferred embodiment of the invention provides a composition comprising Urolithin A, Phytoceramides, lycopene, co-enzyme Q₁₀ and further inactive carriers.

A preferred embodiment of the invention provides a composition comprising Urolithin A, Phytoceramides, zinc, co-enzyme Q₁₀ and further inactive carriers.

A preferred embodiments of the invention provide a composition comprising Urolithin A, lycopene, zinc, co-enzyme Q₁₀ and further inactive carriers.

A preferred embodiment of the invention provides a composition comprising Urolithin A, Phytoceramides, lycopene, zinc, and further inactive carriers.

A preferred embodiment of the invention provides a composition comprising Urolithin A, Phytoceramides, lycopene, zinc,

A preferred embodiment of the invention provides a composition comprising Urolithin A, Phytoceramides, lutein, zinc and, optionally, further inactive carriers.

Another preferred embodiment of the invention provides a composition comprising Urolithin A, Phytoceramides, lutein, zinc, astaxantin and, optionally, further inactive carriers.

An additional preferred embodiments provides a composition of the invention comprising Urolithin A in combination with zinc.

Another preferred embodiment of the invention provides Urolithin A in combination with astaxanthin.

The lycopene may be incorporated into any one of the described compositions in place of lutein. Alternatively, the lutein may incorporated into any one of the described compositions in place of lycopene.

### Preferred Ranges of the Ingredients Described Above are:—

Urolithin A—about 250 mg to about 2500 mg;
Phytoceramides—about 1 mg to about 50 mg;
Lycopene—about 20 mg to about 100 mg, preferably about 20 mg to about 40 mg;
Zinc—about 2 mg to about 50 mg, preferably about 5 mg to about 50 mg;
Co-enzyme Q₁₀—about 50 mg to about 200 mg;
Lutein—about 2 mg to about 50 mg;
Astaxanthin—about 2 mg to about 50 mg.

### Even More Preferred Ranges of the Ingredients Described Above are:—

Urolithin A—about 500 mg;
Phytoceramides—about 20 mg to about 40 mg, preferably 20 mg or 40 mg;
Lycopene—about 20 mg to about 40 mg, preferably 20 mg or 40 mg;
Zinc—about 2.5 mg to about 10 mg, preferably about 2.5 mg or about 5 mg to 10 mg,
more preferably 10 mg;
Co-enzyme Q₁₀—about 150 mg;
Lutein—about 2 mg to about 50 mg, preferably about 5 mg or about 10 mg.
Astaxanthin—about 2 mg to about 10 mg, preferably about 6 mg or 10 mg.

According to a preferred embodiment of the present invention, a composition of the invention comprises Urolithin A in the weight range of about 250 to about 2500 mg, a formulated phytoceramide in the weight range about 1 mg to about 50 mg, lutein in the weight range of about 1 mg to about 50 mg, zinc in the weight range of about 2-50 mg and, optionally, further inactive carriers.

More preferably, a composition of the invention comprises Urolithin A in the weight range of about 500 mg, a formulated phytoceramide in the weight range about 20 mg, lutein in the weight range of about 5 mg, zinc in the weight range of about 2.50 mg and, optionally, further inactive carriers.

According to a preferred embodiment of the present invention, a composition of the invention comprises Urolithin A in the weight range of about 250 to about 2500 mg, a formulated phytoceramide in the weight range about 1 mg to about 50 mg, lutein in the weight range of about 1 mg to about 50 mg, zinc in the weight range of about 2-50 mg, astaxanthin in the weight range of about 2 mg to 50 mg and, optionally, further inactive carriers.

More preferably, a composition of the invention comprises Urolithin A in the weight range of about 500 mg, a formulated phytoceramide in the weight range about 40 mg, lutein in the weight range of about 10 mg, zinc in the weight range of about 5 mg, astaxantin in the weight range of about 6 mg and, optionally, further inactive carriers.

Preferably, the phytoceramides are formulated with cyclodextrins, for example, Ceral OK®.

The lutein is preferably formulated lutein, for example, Lutamax 2020 Free.

Preferably, the zinc is zinc bysglycinate.

Preferably, the astaxantin is natural astaxanthin complex 10% (Asta Rreal L10).

### Uses of Compositions of the Invention

In a further embodiment of the invention, there is provided a composition of the invention for use in a method of prevention, management and/or treatment of a skin condition, disease or disorder. Such uses in skin conditions, diseases or disorders include use in both pathological and non-pathological conditions and cosmetic indications.

In a further embodiment of the invention, skin conditions, diseases or disorders include:
(a) skin aging,
(b) age spots;
(c) liver spots;
(d) dry skin;
(e) radiation induced skin damage (for example, IR, UV, alpha particles, beta particles of gamma irradiation);
(f) lentigo;
(g) hyperpigmentation, for example age-related hyperpigmentation of the skin, or post-inflammatory hyperpigmentation;
(h) melasma (chloasma/mask of pregnancy);
(i) skin irritation, for example, dermatitis;
(j) skin infection, for example, warts;
(k) inflammatory skin conditions (for example, atopic eczema, seborrhoeic eczema, polymorphous photodermatosis, psoriasis, vitiligo),
(l) uneven skin colour (smoothing out thereof),
(m) fine lines and/or wrinkles;
(n) linea nigra;
(o) melanosis; and/or
(p) endocrine diseases, such as Addison's and Cushing's syndrome.

In further embodiments of the invention, there is provided a composition of the invention for use to manage or maintain skin health and/or reduce the impact of chronological aging on biological aging. In a further embodiment, there is provided use of a composition of the invention for prevention or management of aging skin.

In a further embodiment of the invention, there is provided a composition of the invention for use in the treatment of a skin condition, disease or disorder is a skin condition, disease or disorder associated with inadequate mitochondrial activity.

In some embodiments, the skin disease, disorder or condition is selected from the group consisting of melasma, chloasma, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome.

In a further embodiment of the invention, the skin condition, disease or disorder is melasma and is selected from the group consisting of stress-related melasma, pregnancy-related melasma, hypothyroidism-associated melasma, melasma associated with administration of an active ingredient, melasma associated with exposure to sunlight and/or UV light, and melasma associated with exposure to a chemical agent.

As mentioned above, the composition of the invention may be for use in a method of prevention of a skin condition, disease or disorder. That is to say that it is suitable for use by subjects with skin prone to the ailments mentioned herein, for example subjects with intermittent skin issues, for example, skin prone to sunburn, prone to acne and prone to inflammation. The compositions finds use in the management and prevention of the conditions.

In a further embodiment of the invention, there is provided a composition of the invention for the treatment of a condition selected from acne, eczema, psoriasis and rosacea.

In a further embodiment of the invention, there is provided a composition of the invention for the prevention and treatment of skin prone to condition selected from acne, eczema, psoriasis and rosacea.

In a further embodiment of the invention, there is provided the use of composition of the invention for the maintaining or enhancing skin health in a generally healthy subject.

In a further embodiment of the invention, there is provided a non-therapeutic method for preventing or managing a mitochondria-related condition associated with altered mitochondrial function or reduced mitochondrial density, comprising administration of a composition of the invention.

In a further embodiment, there is provided a composition of the invention for use in increasing or maintaining mitochondrial biogenesis.

According to a further embodiment of the invention, there is provided use of a composition of the invention for one or more of the following uses:
a) improving skin health;
b) for maintaining or enhancing skin energy;
c) for maintaining or enhancing skin collagen;
d) reducing skin biological aging;
e) supporting healthy skin aging;
f) reducing skin wrinkles, and/or fine lines;
g) improving skin mitochondrial function;
h) improving skin hydration;
i) promoting replenishment of skin ceramide levels;
j) improving skin elasticity and barrier function;
k) protecting against free radical damage; and/or
I) improving skin dryness;
m) improving photodamaged skin; and/or
n) Improving skin health in skin prone to redness, inflammation, acne or dryness.

According to a further embodiment of the invention, there is provided use of a composition of the invention for one or more of the following uses:
a). maintaining or enhancing skin health and appearance;
b). maintaining or improving skin hydration;
c). enhancing skin elasticity;
d). reducing wrinkles and fine lines;
e). for improving collagen organization in the skin;
f). for boosting collagen production;
g). supporting healthy skin aging;
h). helping replenish ceramide stores;
i). helping protect against free radical damage;
j). support for sun-damage
k). use as an antioxidant;
l). maintaining or promoting healthy skin, hair and nails;
m). improving or maintaining skin barrier function;
n). promoting skin rejuvenation/regeneration; and/or
o). skin barrier support.

According to a further embodiment of the invention, there is provided use of a composition of the invention for one or more of the following:
a). supporting the immune system,
b). supporting the body's defences, and/or
c). maintaining or enhancing immune function.

According to a further embodiment of the invention, there is provided use of a composition of the invention for one or more of the following:
a). reducing skin redness and irritation;
b). as an anti-inflammatory agent; and/or
c). as an antibacterial agent;
In a further embodiment of the invention, there is provided composition of the invention, for one or more of the following uses:
(a) skin bleaching and/or lightening skin colour and/or lightening skin tone;
(b) skin whitening;
(c) protection of skin caused by damage by the environment (For example, damage caused by sunlight/UV irradiation and/or damage caused by pollution);
(d) decreasing pigmentation;
(e) suppressing melanin production.

In a further embodiment of the invention, there is provided a composition of the invention, for example, a composition comprising urolithin A, for one or more of the following uses:
a) improving skin healthspan; and/or
b) improving skin longevity.

### Uses of Composition of the Invention in Hair

Compositions of the invention find use in the treatment or prevention of diseases, disorders and conditions associated with hair. Such uses in diseases, disorders and conditions include use in both pathological and non-pathological conditions and cosmetic indications.

According to a further aspect of the invention there is provided a composition of the invention for stimulating hair growth.

According to a further aspect of the invention there is provided a composition of the invention for preventing or ameliorating hair loss.

According to a further aspect of the invention there is provided a composition of the invention for delaying the onset of hair loss.

According to a further aspect of the invention there is provided a composition of the invention for treating hair thinning.

According to a further aspect of the invention there is provided a composition of the invention for use in a method of increasing hair thickness (i.e. number of hair fibres per surface area) and/or number hair follicles actively producing hair fibres.

According to a further aspect of the invention there is provided a composition of the invention for use in the manufacture of medicament for the treatment of hair loss.

According to a further aspect of the invention there is provided a composition of the invention for use in the manufacture of a medicament for stimulating hair growth.

According to a further aspect of the invention there is provided a composition of the invention for use in the manufacture of a medicament for preventing or ameliorating hair loss.

According to a further aspect of the invention there is provided a composition of the invention for use in the manufacture or a medicament for delaying the onset of hair loss.

According to a further aspect of the invention there is provided a composition of the invention use in the manufacture of a medicament for use in the treatment of hair thinning.

According to a further aspect of the invention there is provided a composition of the invention for use in the manufacture of a medicament for use in a method of increasing hair thickness (i.e. number of hair fibres per surface area) and/or number hair follicles actively producing hair fibres.

According to a further aspect of the invention there is provided a composition of the invention for slowing or preventing premature greying of hair.

According to a further aspect of the invention there is provided a method of treating hair loss in a subject comprising administering to the subject an effective amount of a composition of the invention.

According to a further aspect of the invention there is provided a method of stimulating hair growth in a subject comprising administering to the subject an effective amount of a composition of the invention.

According to a further aspect of the invention there is provided a method of preventing or ameliorating hair loss in a subject comprising administering to the subject an effective amount of a composition of the invention.

According to a further aspect of the invention there is provided a method of delaying the onset of hair loss in a subject comprising administering to the subject an effective amount of a composition of the invention.

According to a further aspect of the invention there is provided a method of treating hair thinning in a subject comprising administering to the subject an effective amount of a composition of the invention.

According to a further aspect of the invention there is provided a method of increasing hair thickness (i.e. number of hair fibres per surface area) and/or number hair follicles actively producing hair fibres in a subject comprising administering to the subject an effective amount of a composition of the invention.

In a further embodiment of the invention, hair diseases, disorders and conditions are selected from: hair loss.

In the present disclosure, the hair-loss includes at least one selected from a group consisting of denutrition-based alopecia, endocrine disorder-based alopecia, vascular disorder-based alopecia, alopecia premature, traction alopecia, alopecia areata, alopecia neurotica, pityriasis alopecia, Trichotillomania, alopecia maligna, female pattern alopecia, male pattern alopecia, androgenetic alopecia, telogen effluvium, tinea capitis, alopecia totalis hypotrichosis, genetic hypotrichosis simplex, systemic drug for alopecia-based hair-loss, mechanical hair-loss, traumatic alopecia, pressure alopecia, anagen effluvium, pityriasis alopecia, alopecia syphilltica, lopecia seborrheica, symptomatic alopecia, alopecia cicatrisata, and alopecia congenita. However, the hair-loss should be understood as meaning including all symptoms classified as the alopecia in this field, regardless of the direct or indirect cause of the occurrence of the hair-loss.

According to a further aspect of the invention there is provided a combination or composition of the invention for the treatment of hair loss, wherein the hair loss is caused by one or more of the following: skin disorders, a medicine, a disease, autoimmunity, iron deficiency, severe stress, scalp radiation, pregnancy or pulling at your own hair.

In a further embodiment of the invention, there is provided a composition of the invention for example, a composition comprising urolithin A, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) promoting new hair growth;
(h) hair maintenance;
(i) scalp health improvement,
(j) improving or maintaining hair strength,
(k) or improving survival of hair transplants; and/or
(l) hair growth restoration promotion.

In a further embodiment of the invention, there is provided a composition of the invention for example, a composition comprising urolithin A, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

In a further embodiment of the invention, there is provided the use of composition of the invention for the maintaining or enhancing hair health in a generally healthy subject.

### Uses of Composition of the Invention in Nails

Compositions of the invention find use in the treatment or prevention of diseases, disorders and conditions associated with nails. Such uses in diseases, disorders and conditions include use in both pathological and non-pathological conditions and cosmetic indications.

In a further embodiment of the invention, nail diseases, disorders and conditions include nail deformities (changes in nail shape) and nail dystrophies (changes in nail texture, colour or both.

Nail conditions include: brittle nails, triangular worn down nails, trachyonychia, and habit tic deformity

In a further embodiment of the invention, there is provided the use of composition of the invention for the maintaining or enhancing nail health in a generally healthy subject.

### Further Use of Compositions of the Invention

Biotin has been reported to have utility in other diseases, disorders and conditions, such as diabetes (e.g. diabetes mellitus), syndrome X, obesity, dyslipidemia, multiple sclerosis, demyelinating neuropathies (such as Amyotrophic Lateral Sclerosis, demyelinating neuropathy associated with immunoglobulin M (IgM) monoclonal gammopathy and antibodies against myelin-associated glycoprotein (MAG), typical chronic inflammatory demyelinating polyradiculoneuropathy, atypical chronic inflammatory demyelinating polyradiculoneuropathy, Charcot Marie Tooth Ia disease, Guillain-Barre syndrome and neuromyelitis optica), X-linked adrenoleukodystrophy (for example, adrenomyeloneuropathy), visual impairment or atrophy, autism spectrum disorder. Huntington's disease. Therefore, in a further aspect of the invention there is provided a composition of the invention for the treatment or prevention of a disease, disorder or condition, selected from diabetes (e.g. diabetes mellitus), syndrome X, obesity, dyslipidemia, multiple sclerosis, demyelinating neuropathies (such as Amyotrophic Lateral Sclerosis, demyelinating neuropathy associated with immunoglobulin M (IgM) monoclonal gammopathy and antibodies against myelin-associated glycoprotein (MAG), typical chronic inflammatory demyelinating polyradiculoneuropathy, atypical chronic inflammatory demyelinating polyradiculoneuropathy, Charcot Marie Tooth Ia disease, Guillain-Barre syndrome and neuromyelitis optica), X-linked adrenoleukodystrophy (for example, adrenomyeloneuropathy), visual impairment or atrophy, ungual pathologies, autism spectrum disorder, Huntington's disease.

### Compounds of Formula (I) (Urolithins)

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

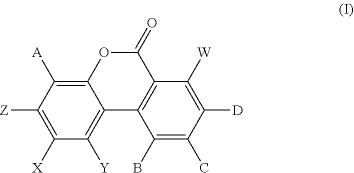

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the combinations of the invention.

In one aspect of a combination of the invention, a suitable compound is a compound of Formula (I) wherein A, C, D and Z are independently selected from H and OH and B, W, X and Y are all H, preferably at least one of A, C, D and Z is OH.

Particularly suitable compounds are the naturally occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

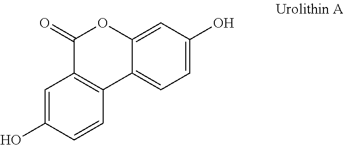 [SMILES: O=c1oc2cc(O)ccc2c2ccc(O)cc12]

According to one embodiment there is provided a combination, composition, use or method of the invention wherein the compound of formula (I) is urolithin A.

According to one embodiment there is provided a combination, composition, use or method of the invention wherein the compound of formula (I) is urolithin B.

According to one embodiment there is provided a combination, composition, use or method of the invention wherein the compound of formula (I) is urolithin C.

According to one embodiment there is provided a combination, composition, use or method of the invention wherein the compound of formula (I) is urolithin D.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

The urolithin used in compositions of the invention is preferably a high purity urolithin, for example a high purity urolithin A. For example, the urolithin in the composition has a purity of over 95% w/w, for example >97% w/w, for example >97.5% w/w, for example >98% w/w, for example >98.5% w/w, for example >99% w/w, for example >99.5% w/w, for example >99.75% w/w. The composition thus contains less than 5% w/w of urolithin by-products or synthetic/preparative intermediates; for example the composition contains <3% w/w of urolithin by-products or synthetic/preparative intermediate, for example <2% w/w, for example <1% w/w of urolithin by-products or synthetic/preparative intermediates, for example <0.5% w/w of urolithin by-products or synthetic/preparative intermediates, for example <0.25% w/w of urolithin by-products or synthetic/preparative intermediates.

### Urolithin Administration/Dosage Regimes

Compositions of the invention may be administered by any suitable method, for example, orally or topically.

### Oral Administration

In one embodiment, the administration of compositions of the present invention preferably involves oral administration of a urolithin of formula (I) or salt thereof to a subject in a daily amount in the range of about 1.1 to about 8.8 mmol, for example, from about 1.7 to about 6.0 mmol per day, for example, from about 1.7 to about 2.7 mmol per day, or from about 2.8 to about 6.0 mmol per day. As discussed below, administration is preferred in the range 125 mg to 2000 mg urolithin A (which corresponds to about 0.55 to 8.8 mmol), 250 mg to 2000 mg urolithin A (which corresponds to about 1.1 to 8.8 mmol), for example 250 mg to 1500 mg, such as 250 mg to 1000 mg, which results in a surprisingly good pharmacokinetic profile. In one embodiment the dose is 125 mg/day, in an alternative embodiment the dose is 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
125 mg once or twice a day;
250 mg once or twice a day;
500 mg once or twice a day;
750 mg once or twice a day;
1000 mg once or twice a day;
1250 mg once or twice a day; or
1500 mg once or twice a day.

The uses and methods of the present invention involve daily administration of the compound of formula (I) or salt thereof, or of a composition comprising the compound or salt. In some embodiments, the compound or composition is administered once per day, i.e. the compound or composition is to be administered at least once per 24-hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

In some embodiments, the method comprises administering the compound or salt daily for a period in the range of from 7 days to 6 months, from 14 days to 6 months, from 14 days to 5 months, from 7 days to 4 months, from 7 days to 3 months, from 7 days to 2 months, from 7 days to 1 month. In a further embodiment, the method comprising administration of a composition of the invention on an ongoing basis.

The uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from about 100 mg to about 600 mg/day for example, from about 125 mg to about 600 mg/day for example, about 200 mg to about 600 mg per day, for example from about 300 mg to about 600 mg/day, from 400 to 600 mg/day or an amount of urolithin A in said ranges twice per day. In a preferred embodiment the method involves administration of urolithin A in an amount in the range of from 450 to 550 mg, more preferably approximately 500 mg per day or twice per day. In a further preferred range, the method involves administration of urolithin A in the range from about 200 mg to about 300 mg, more preferably about 250 mg per day or twice per day. In a yet further preferred range, the method involves administration of urolithin A in the range from about 100 mg to about 150 mg, more preferably about 125 mg per day or twice per day.

In other embodiments the methods involve administration of urolithin A in an amount in the range of from 50 to 1000 mg/day twice per day, or in the range of from 50 to 750 mg, or in the range of from 100 to 500 mg, or in the range of from 150 to 350 mg, for example 250 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

In other preferred embodiments, the uses or methods involve administering urolithin A to the subject in an amount in the range of from 1.8 to 7.1 mg/kg/day, such as 2.5 to 6.5 mg/kg/day. In another embodiment, the uses or methods involve administering urolithin A to the subject in an amount in the range of 3 to 7 mg/kg/day. In another embodiment, the uses or methods involve administering urolithin A to the subject in an amount in the range of from 3.0 to 5.0 mg/kg/day.

In other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

Dosage regimes which combine two or more of a 125 mg, a 250 mg, a 500 mg dose and a 1000 mg dose may be advantageous. For example, a twice daily dosage regime which combines a first dose of 1000 mg and a second dose several hours later of 500 mg 500 mg or a first dose of 500 mg and a second dose hours later of a 250 mg dose, or a first dose of 250 mg and a second dose hours later of a 125 mg dose. Said 125 mg, 250 mg or 500 mg dose may be 6-18 hours after the 250 mg, 500 mg or 1000 mg dose, for example 8-12 hours after the 1000 mg dose. For example, about 12 hours after the 1000 mg dose. Thus, according to a further aspect of the invention there is provided the treatment of a disease, disorder or condition with a compound of Formula (I) which comprises a twice daily dosage regime comprising a first dose of 250 mg or 500 mg or 1000 mg, followed by a second dose of 125 mg or 250 mg or 500 mg wherein the two doses are separated by 6-18 hours.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided a composition wherein the compound of formula (I) is administered at a dose of about 1.7-28.6 mg/kg, for example about 1.7 to 15 mg/kg. for example about 3.5-14.0 mg/kg, for example 5.0-10.0 mg/kg.

### Topical Administration

In a further embodiment of the invention, when the administration is topical administration, there is provided a composition of the invention, comprising:
(a) about 0.1% (w/w) to about 5% (w/w) of a compound of formula (I), or a salt thereof, for example, urolithin A, or a salt thereof.

In a further embodiment of the invention, when the administration is topical administration, there is provided a composition of the invention, comprising:
(a) about 0.1% (w/w) to about 5% (w/w) of a compound of formula (I), or a salt thereof, for example, urolithin A, or a salt thereof.

In a further embodiment the concentration range of a compound of formula (I), or a salt thereof, for example, urolithin A, or a salt thereof, for topical administration is from about 0.8% (w/w) to about 4% (w/w) of the composition, for example, about 0.8% (w/w) to about 3% (w/w) of the composition, for example, about 0.8% (w/w) to about 2% w/w of the composition, for example about 1% (w/w) to 2% w/w of the composition, for example about 1% (w/w) to about 1.5% w/w, for example about 1% w/w, such as about 0.8% (w/w), or about 0.9% (w/w), or about 1% (w/w), or about 1.5% (w/w) or about 2% (w/w) or about 2.5% (w/w) or about 3% (w/w) or about 4% (w/w) of the composition. In a further embodiment of the invention, the concentration range of a compound of formula (I), or a salt thereof, for example, urolithin A, or salt thereof, is about 1% (w/w) to about 5% w/w, for example, about 2% (w/w) to about 5% w/w, for example, about 3% (w/w) to about 5% w/w, such as about 4% (w/w) to about 5% w/w.

### Animals

Compositions of the invention are suitable for other mammals in addition to humans, scaled appropriately to the size of the non-human mammal. For example, scaled according to the Table 2 below:

| | | | To convert human | |
|---|---|---|---|---|
| | | | dose in mg/kg to | |
| | Reference | To convert dose | AED in mg/kg, either | |
| | body | in mg/kg to | Multiply | Divide |
| | weight | dose in mg/m2, | human | human |
| Species | (kg) | divide by km | dose by | dose by |
| Mouse | 0.02 | 3 | 12.3 | 0.081 |
| Hamster | 0.08 | 5 | 7.4 | 0.135 |
| Rat | 0.15 | 6 | 6.2 | 0.152 |
| Ferret | 0.3 | 7 | 5.3 | 0.189 |
| Guinea Pig | 0.4 | 8 | 4.6 | 0.216 |
| Rabbit | 1.8 | 12 | 3.1 | 0.324 |
| Cat | 2 | 11.7 | 3.2 | 0.316 |
| Monkey | 3 | 12 | 3.1 | 0.324 |
| Dog | 10 | 20 | 1.9 | 0.541 |
| Marmoset | 0.35 | 6 | 6.2 | 0.162 |
| Squireel | 0.6 | 7 | 5.3 | 0.189 |
| Monkey | | | | |
| Baboon | 12 | 20 | 1.9 | 0.541 |
| Micro pig | 12 | 27 | 1.4 | 0.73 |
| Mini pig | 40 | 35 | 1.1 | 0.946 |

Therefore, according to a further aspect of the invention there is provided a composition for use in a non-human mammal wherein the dose is scaled according to the table above.

### Biotin Administration/Dosage Regimes

Biotin comprises biotin, any of its salts and/or its derivatives that have the same or an equivalent biological functionality to biotin. Biotin derivatives include biotin esters, biotinamides, carboxybiotin, carboxybiotin esters and carboxybiotinamides, including pharmaceutically acceptable salts thereof.

Compositions of the invention comprise between about 5 μg-about 50 mg biotin, or a derivative thereof. In one embodiment the composition comprises about 100 μg to about 25 mg, for example about 1 mg to about 25 mg, such as about 1 mg to about 20 mg, about 1 mg to about 15 mg, about 1 mg to about 12 mg, about 1 mg to about 10 mg or about 2 mg to about 10 mg. In a further embodiment, compositions comprise between about 0.1 mg to about 5 mg, about 5 mg to about 10 mg, about 10 mg to about 15 mg, about 15 mg to about 20 mg or about 20 mg to about 25 mg. In a further embodiment compositions comprise about 500 mg, about 1 mg, about 2 mg, about 3 mg, about 4 mg, about 5 mg, about m6g, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg,

Compositions of the invention may be administered at a dose of between about 0.1 mg to about 50 mg biotin per day, for example, between about 0.5 mg/day to about 50 mg/day, for example about 1 g/day to about 50 mg/day, for example, about 1 mg/day to about 40 mg/day, for about 1 mg/day to about 30 mg/day, for example, about 1 mg/day to about 25 mg/day, for example, about 1 mg/day to about 20 mg/day, for example, about 1 mg/day to about 15 mg/day or about 1 mg/day to about 10 mg/day. In a further embodiment, compositions of the invention are administered at a dose of about 1 mg/day to about 5 mg/day, about 5 mg/day to about 10 mg/day, about 10 mg/day to about 15 mg/day, about 15 mg/day to about 20 mg/day, about 20 mg/day to about 25 mg/day, about 25 mg/day to about 30 mg/day, about 30 mg/day to about 35 mg/day, about 35 mg/day to about 40 mg/day, about 40 mg/day to about 45 mg/day or about 45 mg/day to about 50 mg/day. In a further embodiment of the invention, compositions of the invention are administered at a dose of about 1 mg/day, about 2.5 mg/day, about 5 mg/day, about 7.5 mg/day, about 10 mg/day, about 15 mg/day, about 20 mg/day, about 25 mg/day, about 30 ng/day, about 40 mg/day or about 50 mg/day.

Topical compositions of the invention comprise about 0.1% to about 10% biotin, for example, about 0.5% to about 10%, for example, 0.5% to about 8%, for example, 0.5% to 5% or 1% to 5%. In a further embodiment, topical compositions comprise about 0.5% biotin, about 1%, about 2%, about 3%, about 4%, or about 5%

The composition of the present invention contains biotin in an amount sufficient to administer to a subject a dosage from about 0.01 mg to about 0.6 mg per kg body weight per day, for example, from about 0.01 mg to about 0.35 mg per kg body weight per day, such as from about 0.01 mg to about 0.2 mg per kg body weight per day or from about 0.01 mg to about 0.15 mg per kg body weight per day.

### Additional/Combination Therapy

Compositions of the invention may comprise one of more further active agents, for use in the treatment or prevention of diseases, disorders or conditions, or for maintaining or enhancing skin health in a generally healthy subject, or for reducing the impact of chronological aging on biological aging.

Any active agent which is known to be useful, or which has been used or is currently being used for the treatment or prevention of diseases, disorders or conditions can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used for the treatment or prevention of diseases, disorders or conditions associated with hair, nails and/or skin.

Examples of further active agents include: carnitine and/or acyl-carnitines, cyclodextrin, (−)-epigallocatechin gallate (EGCG), finasteride, taurine, thiamine, ubiquinone, caffeine, collagen, Vitamin B3 (niacin, niacinamide), zinc salts and/or saw palmetto.

The composition of the invention described herein may be taken orally. Such a composition exerts its effects by systemic distribution of some or all of the active component(s) and subsequent beneficial effects in the skin. When using the composition, a subject may also apply a topical treatment to the skin. Such a topical treatment may also contain a compound of formula (I). That is to say that a possible combination treatment may include the administration of the same active, a compound of formula (I) (for example Urolithin A) by two different routes.

### Aging/Healthspan

According to a further embodiment of the invention, there is provided a composition of the invention for use in the increasing or prolonging of healthspan. Healthspan can be defined as the part of a person's life during which they are generally in good health. In a further embodiment of the invention, there is provided a composition of the invention, for slowing aging, for example, increasing skin longevity and skin healthspan.

### Compositions

The uses and methods of the present invention preferably involve oral administration of the compositions of the invention. Any suitable oral composition may be used. Accordingly, the use of a range of compositions which are suitable for oral administration, is envisaged. Thus, in some embodiments, the composition is administered in the form of an oral composition and one or more excipients suitable for oral administration. Oral compositions may comprise compositions having the form of a pill, softgel, tablet, capsule, caplet, lozenge, pastille, granules, powder for suspension, oral solution, oral suspension, oral emulsion, syrup, gummies, or the like.

In a further embodiment of the invention, the composition is administered by any means known to the skilled person for administration such as, intramuscular, sublingual, cutaneous, inhalation and auricular. Oral administration is preferred.

Compositions may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell, for example it may be a soft vegan-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In some preferred embodiments, the use or method comprises administration of the compound of formula (I) or salt thereof (e.g. urolithin A), of a defined particle size distribution. A defined particle size distributionenables the compound of formula (I) to disperse or dissolve more rapidly. A defined particle size distribution can be achieved by methods established in the art, for example compressive force milling, hammer milling, universal or pin milling, or jet milling (for example spiral jet milling or fluidised-bed jet milling) may be used. Jet milling is especially suitable. Alternatively, synthetic methods for preparation of compounds of formula (I) may produce particles of the required particle size distribution If a compound of formula (I) with a defined particle size distribution is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 4.0 to 10 μm, for example 1.5 to 7.5 μm, for example 2.8 to 5.5 μm. Preferably, the compound has a D₉₀ size of under 100 μm. More preferably, the compound has a D₉₀ size of under 75 μm, for example under 50 μm, for example under 25 μm, for example under 20 μm, for example under 15 μm. The compound preferably has a D₉₀ in the range 5 to 100 μm, for example 5 to 50 μm, for example 8 to 25 μm, for example 5 to 20 μm, for example 7.5 to 15 μm, for example 8.2 to 16.0 μm. Preferably, the compound has a D¹⁰ in the range 0.5 to 2.5 μm, for example 0.5 to 2.0 μm, for example 0.5-1.0 μm. Preferably, the compound of formula (I) or salt thereof (e.g. urolithin A) has a D₉₀ in the range 8.2 to 16.0 μm, a D₅₀ in the range 2.8 to 5.5 μm and a D¹⁰ in the range 0.5 to 1.0 μm.

In a further embodiment, the compound of formula (I) or salt thereof has a size distribution selected from one of the following:
(i) D₅₀ size in the range 0.5 to 50 μm and a D₉₀ size in the range 5 to 100 μm,
(ii) D₉₀ size in the range 8.2 to 16.0 μm, a D₅₀ size in the range 2.8 to 5.5 μm and a D₁₀ size in the range 0.5 to 1.0 μm;
(iii) D₉₀ size in the range 8 to 25 μm, a D₅₀ size in the range 4 to 10 μm and a D¹⁰ size in the range 0.5 to 2.0 μm;
(iv) D₅₀ size in the range 0.5 to 20 μm and a D₉₀ size in the range 5 to 50 μm;
(v) D₅₀ size under 50 μm and a D₉₀ size under 75 μm;
(vi) D₅₀ size under 25 μm and a D₉₀ size under 50 μm;
(vii) D₅₀ size under 10 μm and a D₉₀ size under 20 μm;
(viii) D₅₀ size under 10 μm and a D₉₀ size under 15 μm; or
(ix) D₅₀ size of 10 μm and a D₉₀ size of 20 μm.

In one embodiment, the composition is administered in the form of a composition comprising: a) a medium-chain triglyceride; b) the compound of formula (I) or salt thereof; and c) one of more agents selected from agents for improving skin health. Within those embodiments, preferably the compound of formula (I) (e.g. urolithin A) is of a defined particle size distribution. Compositions comprising a urolithin or salt thereof, and a medium chain triglyceride can be found in International patent application: WO2017/036992.

For the avoidance of doubt, compounds in compositions of the invention may be formulated in the same composition or formulated in separate compositions for simultaneous, separate or sequential administration.

### Kits

Also within the scope of the present invention are kits, comprising composition of the invention. Kits typically include a label indicating the intended use of the contents of the kit and instructions for use. The term “label” includes any writing, or recorded material supplied on or with the kit, or which otherwise accompanies the kit.

The invention further provides kits comprising:
(a) a composition of the invention; and
(b) a container, or containers, for containing said composition; and
(c) optionally instructions for simultaneous, separate or sequential administration.

A kit of the invention may include a second composition that is also to be used by the subject as part of the treatment. For example, the second composition may be a topical treatment to apply to the skin. Such a topical treatment may also contain a compound of formula (I). That is to say that a possible kit may include two components each containing the same active (a compound of formula (I) (for example Urolithin A)) for administration by two different routes.

The invention therefore further provides a kit comprising:
(a) a composition of the invention for oral administration; for example, comprising urolithin A,
(b) a topical skin formulation, for example, comprising urolithin A;
(c) a container, or containers, for containing said compositions; and
(d) optionally instructions for simultaneous, separate or sequential administration.

In particular, the invention provides such a kit wherein the topical skin formulation includes a compound of formula (I) as an active ingredient, for example Urolithin A. The invention therefore further provides a kit comprising:
(a) a composition of the invention for oral administration, for example, comprising urolithin A;
(b) a composition of the invention comprising a compound of formula (I) (for example Urolithin A) for topical administration;
(c) a container, or containers, for containing said compositions; and
(d) optionally instructions for simultaneous, separate or sequential administration.

### Preparation of Compositions of the Invention

In a further embodiment, there is provided a process for preparing a composition of the invention, which comprises, mixing
(a) a compound of formula (I), or a salt, prodrug, metabolite or derivative thereof, for example, urolithin A;
(b) one or more agents for improving skin health, as described above and/or one or more further active ingredient(s), and
(c) one or more excipients, for example, one of more excipients suitable for oral administration.

The term ‘about’ refers to a tolerance of ±20% of the relevant value, for example ±15% of the relevant value, such as ±10% of the relevant value or ±5% of the relevant value.

The term ‘biotin derivative’ refers to any derivatives that have the same or an equivalent biological functionality as biotin. Examples of biotin derivatives include: biotin esters, biotinamides, carboxybiotin, carboxybiotin esters and carboxybiotinamides, including pharmaceutically-acceptable salts thereof.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating nutrient or drug absorption, reducing viscosity or enhancing solubility.

The term ‘hair-loss’ refers to a phenomenon in which hair is lost from the skin or a state in which hair is sparse or thin and is a term that may be used interchangeably with alopecia. In the present invention, the hair includes hair roots and hair follicles on the head, hairs on the head, eyelashes and eyebrows, beards, armpit hair, pubic hair, and hair roots and hair follicles throughout the body.

The term “pharmaceutically acceptable” means approved by a regulatory agency of the Federal or a state government or listed in the U.S. Pharmacopeia or other generally recognized pharmacopeia for use in animals, and more particularly in humans, or a GRAS additive (Generally Recognized As Safe).

The term, “separate” administration means the administration of each of two or more compounds to a subject from non-fixed dose dosage forms simultaneously, substantially concurrently, or sequentially in any order. There may, or may not, be a specified time interval for administration of each the compounds.

The term “sequential” administration means the administration of each of two or more compounds to a subject from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a subject in a single action such as where each compound is administered independently at substantially the same time or separately within time intervals that allow the compounds to show’ a cooperative therapeutic effect.

The term “therapeutically effective amount” as used herein refers to the amount of a compound or compounds that, when administered, is sufficient to assist in the prevention of the development of, or to relieve to some degree, one or more symptoms of a condition. The particular dose of each compound administered according to this invention will of course be determined by the particular conditions surrounding the case, including the compound administered, the route of administration, the particular condition being treated, as well as considerations such as age, weight and sex of the treated subject. The term ‘therapeutically effective amount’ also refers to the effective dose when a composition of the invention is being used for a non-disease condition, i.e. when used for non-medical uses.

The invention will now be illustrated with respect to the following non-limiting examples

### EXAMPLES

### Example 1: Representative Powder or Gel Compositions with Benefits for Skin Health

1a. Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramides¹ | 1-50 | mg | 40 | mg |
| Lycopene | 20-100 | mg | 40 | mg |
| Zinc | 5-50 | mg | 10 | mg |
| Co-enzyme Q10 | 50-200 | mg | 150 | mg |
| Optionally Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1b. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramides¹ | 1-50 | mg | 40 | mg |
| Optionally Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1c. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Lycopene | 20-100 | mg | 40 | mg |
| Optionally Astaxanthin | 1-30 | mg | 6 | mg |
| Further inactive carriers | | | | |

1d. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Zinc in salt form | 5-50 | mg | 10 | mg |
| Further inactive carriers | | | | |

1e. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Astaxanthin | 1-30 | mg | 6 | mg |
| Further inactive carriers | | | | |

1f. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramides¹ | 1-50 | mg | 40 | mg |
| Lycopene | 20-100 | mg | 40 | mg |
| Optional Co-enzyme Q10 | 50-200 | mg | 150 | mg |
| Optional Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1g. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramides¹ | 1-50 | mg | 40 | mg |
| Zinc | 5-50 | mg | 10 | mg |
| Optional Co-enzyme Q10 | 50-200 | mg | 150 | mg |
| Optional Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1h. Further Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Lycopene | 20-100 | mg | 40 | mg |
| Zinc | 5-50 | mg | 10 | mg |
| Optional Co-enzyme Q10 | 50-200 | mg | 150 | mg |
| Optional Astaxanthin | 1-30 | mg | 6 | mg |
| Further inactive carriers | | | | |

1i. Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramide¹ | 1-50 | mg | 40 | mg |
| Lycopene | 20-40 | mg | 40 | mg |
| Zinc | 5-50 | mg | 10 | mg |
| Optional Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1j. Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramide¹ | 1-50 | mg | 40 | mg |
| Lycopene | 20-100 | mg | 20 | mg |
| Zinc | 5-50 | mg | 5-10 | mg |
| Optional Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)

1k. Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramide¹ | 1-50 | mg | 20 | mg |
| Lutein² | 1-50 | mg | 5 | mg |
| Zinc³ | 2-50 | mg | 2.5 | mg |
| Optional Astaxanthin | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)
²Lutein in the table above refer to formulated lutein, for example, Lutamax 2020 Free.
³Zinc bysglycinate (27% zinc) is preferably used.

1l. Formulation of Urolithin A with Benefits for Skin Health

| | | |
|---|---|---|
| Component | Weight range | Weight |

| | | | | |
|---|---|---|---|---|
| Urolithin A | 250-2500 | mg | 500 | mg |
| Phytoceramide¹ | 1-50 | mg | 40 | mg |
| Lutein² | 1-50 | mg | 10 | mg |
| Zinc³ | 2-50 | mg | 5 | mg |
| Astaxanthin⁴ | 1-30 | mg | 6 | mg |
| Further inactive carriers | | | | |

¹Phytoceramides in the table above refer to formulated phytoceramides, for example, formulated phytoceramides comprising cyclodextrins, for example, CeraLOK ® (Anderson Advanced Ingredients, Irvine, CA, USA)
²Lutein in the table above refer to formulated lutein, for example, Lutamax 2020 Free.
³Zinc bysglycinate (27% zinc) is preferably used.
⁴Natural Astraxanthin Complex 10% (Asta Real L10)

The formulations according to Examples 1a to 11 above may be provided as a soft gel (for example in a capsule having a shell), as a gummy, as a powder as a tablet, as a lotion or as a cream.

### Example 2. Impact on Skin Aging of a Combination of a Topical and Oral Formulations Comprising Urolithin A

The aim of this study is to investigate the impact on skin aging of a combination of a topical and oral product in comparison to the respective group receiving an oral placebo combined with the topical product. The topical product is a cosmetic designed to nourish the skin from the outside and the oral product is a food supplement improving skin health from within. Both products contain Urolithin A. The oral product is the Formulation of 1k above.

The topical product is a cream formulation with UA 1%. The cosmetic product contains an active on skin aging and is acting on biological hallmarks of skin aging. The food supplement also contains several key bioactives designed to target different hallmarks of skin aging systemically.

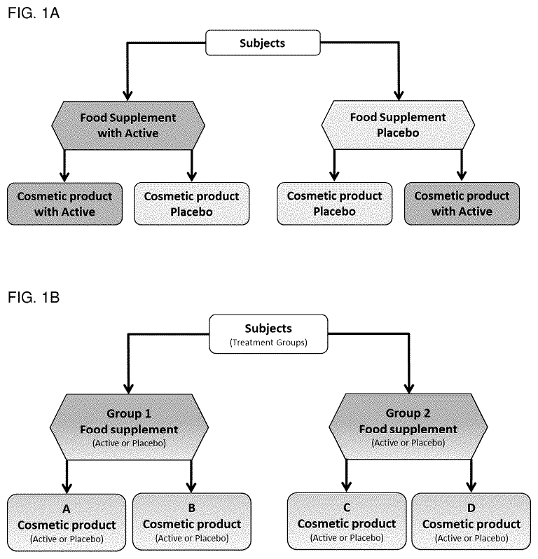

For this study female subjects aged preferably between 45 and 65 years with visible wrinkles in the face will be enrolled and divided into two treatment groups. One group will be assigned to the treatment group testing the active food supplement in combination with the active and placebo cosmetic test product in split-face design. The other group will be assigned to the treatment group testing the placebo food supplement in the same combination with the active and placebo formulations of the cosmetic test product in split-face design (see FIG. 1A).

Application of the cosmetic test products will be in the face and on the volar forearms over a period of 12 weeks. Three days before reaching the 6 and 12 weeks application period, application of the placebo cosmetic product will be stopped and continued after interim assessment visit. As compliance with a 12 week untreated side of the face is difficult, this 3 days interruption period allows the profilometry and hydration effect of the placebo to fade away and enables to draw a statement as if the test areas had been untreated.

Anti-wrinkle efficacy will be assessed in the periorbital regions by investigating the three-dimensional structure of the wrinkles (DermaTOP). Skin hydration, skin elasticity effects and skin barrier function will be investigated by Corneometer, Cutometer and Tewameter measurements. Images for wrinkle analysis and for documentation purpose will be taken with VISIA-CR. All instrumental measurements will be done at the beginning as well as after 6 and after 12 weeks of application.

To analyze mitochondrial effects biopsies will be taken from a subpanel of both treatment groups on volar forearms prior and after 12 weeks of product application and will be analyzed for biomarkers by 3rd parties.

A questionnaire regarding product traits will be filled in after 6 and 12 weeks of product application.

### Main Endpoints:

Change from baseline in anti-wrinkles efficacy (assessed on three-dimensional structure of the wrinkles in the periorbital regions) after 6 and 12 weeks

### Additional Outcomes:

Change from baseline in skin barrier function (assessed on transepidermal waterloss) after 6 and 12 weeks.
Change from baseline in skin hydration (assessed on skin capacitance) after 6 and 12 weeks.
Change from baseline in skin firmness after 6 and 12 weeks
Change from baseline in skin elasticity after 6 and 12 weeks
Change from baseline for wrinkles and fine lines (assessed on VISIA-CR Image. analysis) after 6 and 12 weeks.
Change from baseline in skin hallmarks of aging (assessed on skin biopsies) after 12 weeks in a subpanel.
Subjective evaluation of product traits assessed via questionnaire after 6 and 12 weeks

The study will be reviewed by an independent institutional review board (IRB) for ethical approval.

### 1.1 Efficacy Assessment

The following efficacy assessment(s) will be performed:
Skin roughness-Ra by DermaTOP [μm]
Skin roughness-Rz by DermaTOP [μm]
Transepidermal water loss (TEWL) by Tewameter [g/(m²h)]
Skin hydration-Skin capacitance by Corneometer [a.u.]
Skin Firmness-RO (Uf) by Cutometer [mm]
Skin Elasticity-R7 (Ur/Uf) by Cutometer
Wrinkle and fine lines analysis based on VISIA-CR images.
Questionnaire of product traits.
Facial photography by VISIA-CR
Biomarker analysis for skin aging features in 3 mm punch-biopsies

### 1.2 Tolerability Assessment

Not applicable

### 1.3 Type of Product(s)

Food supplement (with active and placebo)
Cosmetic care product for the face (with active and placebo)

### 1.4 Study Outline

Exploratory
Randomized
Double blind for the food supplement
Blind for subjects for the cosmetic product
Intra-Individual comparison (before and after treatment)
Inter-individual comparison between treatment groups
Placebo controlled

### 1.5 Type of Statistics

Comparison between treatment groups
Comparison between assessment times

### 1.6 Assessment Times

Baseline (Day 1)
Day 43 (After 6 weeks of application)
Day 85 (After 12 weeks of application)

### 2.0 Test Materials

FIG. 1B shows the different treatment groups, as described below.
1A Food supplement (active/placebo) combined with the cosmetic product (active/placebo)
1B Food supplement (active/placebo) combined with the cosmetic product (active/placebo)
2C Food supplement (active/placebo) combined with the cosmetic product (active/placebo)
2D Food supplement (active/placebo) combined with the cosmetic product (active/placebo)

The test material(s) will be food supplement and cosmetic products, as furnished by the sponsor. Test and control materials, if applicable, will be identified by a study site code (e.g. “A”, “B” etc.) and/or by a sponsor identification code in a separate delivery form. Test materials will be used undiluted or diluted, as specified in the delivery form. The sponsor will identify potential hazard of the test materials furnished by the sponsor or his designee(s) associated with this study. It will be the responsibility of the sponsor to determine, for each batch of product, the identity, strength, purity, composition and other characteristics that appropriately define the test substances before their use in the study. The determination of the stability of the test substances and documentation of methods of synthesis or derivation are also the sponsor's responsibility. The test materials will be stored at room temperature in the containers in which they are received unless otherwise specified in the delivery form. Test material remaining at the conclusion of the study will be destroyed at least 6 weeks after issuance of the final report unless requested otherwise.

### 2.1 Labelling and Storage

A total of 120 HDPE bottles of each food supplement with active and placebo and a total of 200×50 ml pots of the cosmetic product with active and 250×50 ml pot of the placebo cosmetic product will be supplied by sponsor.

The label for the food supplement will be as follow:

### Food Supplement

### Instruction for use:

Take 2 soft gels a day with a large glass of water.
Do not exceed the recommended daily dose.
Use for clinical purposes only.
Keep away from children.
Follow the information provided carefully.
Store in a cool, dry place away from light and moisture.

### 2.2 Test Area

Face
Volar forearm

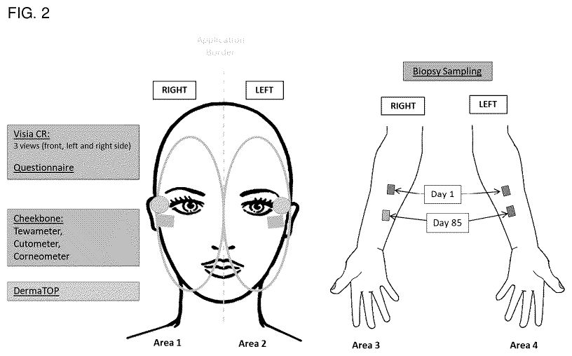

### 2.3 FIG. 2 Shows the Assignment of Treatment Groups and Test Areas on a Subject's Face and Forearms.

### Randomization

Subjects will be divided equally in 2 treatment groups. Group 1 will receive treatments A and B and group 2 C and D. Treatments will be randomly and balanced assigned to left and right test area in each group.

Biopsy extraction will be performed on a subpanel of 12 randomly chosen subjects per treatment group, in total of 24 subjects. The treatments of the forearms in these 24 subjects will be assigned to the same randomisation as for the face.

### 2.4 Application Volume

Approximately a hazelnut-sized amount for each test material to half of the face and to one volar forearm. The correct amount of test products to be applied will be demonstrated by a technician at the study site.

### 2.5 Length of Wash-Out Phase

At least 3 days (the wash-out phase will be between the screening and day 1)

### 2.6 Standard Washing Procedure

The subjects will receive a wash-out product (silicon-free, soap/alkali-free, paraben-free) provided by the study site. The wash-out product will be used at least 3 days before the study start (day1) and throughout the study according to the following instructions:

The wash-out product will be used once daily in the evening directly before test materials application. In the morning the face will be cleaned with water only.

The wash-out product will be used for the face only; no wash-out product will be used on the volar forearms.

### 2.7 Application Mode and Frequency

### Food Supplement:

2 soft gels once per day with a large glass of water in the morning.

The first application of the food supplement will take place on day 2 at home and then applied 12 weeks by the subjects.

The last application will be done on the morning before the last visit to the study site.

### Cosmetic Product:

The cosmetic product will be applied twice daily in the morning and the evening by the subjects at home. The cosmetic product will be used by the subjects themselves, appropriate instructions will be given to the subjects orally and writing.

The first application of the cosmetic product will take place at the study site under the supervision of a technician on day 1. The cosmetic product will then be applied 12 weeks by the subjects at home, according to application training.

On the assessment days (day 43 and 85), subjects will be instructed not to apply the test products in the morning before visiting the study center, the morning application will take place at the site after all assessments.

On day 42 and day 84, subjects will be instructed to apply the test materials the last time in the evening and nothing to apply in the morning before visiting the study site on day 43 and day 85.

Three days before the assessment days (day 43 and 85), subjects will be instructed to stop using with one cosmetic product. It will be communicated at day 1 and day 43 which cosmetic code it will be.

### 2.8 Duration of Treatment

12 weeks per subject

### 2.9 Wash-Out Product

### 2.10 Post-Treatment Product

A post-treatment product (sunscreen for sensitive skin, SPF 50+) will be used to the subpanel with biopsies after the end of study conduct to be used when exposing the biopsy wounds to sunlight.

### 3.0 Study Population

### 3.1 Subject Numbers

A minimum of 72 subjects will be recruited for this test from the general population and neighbouring communities of the study site's location so that about 60 subjects are expected to finish in the study. Subjects who drop-out after randomization into the study will not be replaced. All subjects will have a complete understanding of the test procedure.

### 3.2 Selection of Subjects

All below mentioned inclusion, exclusion criteria and instructions for the subjects will be checked by a questionnaire during the screening as well as before the start of the study and during the study. Conditions developing during the course of the study listed in the exclusion criteria and instructions as well as protocol deviations do not necessarily lead to the subject's exclusion. The investigator decides whether the subject is still eligible.

The subjects will be instructed to inform the study site in case of medical problems or changes in therapies.

### Inclusion Criteria.

Written Informed Consent to participate in the study
Willingness to actively participate in the study and to come to the scheduled visits
Female
From 45 to 65 years of age, in order to include sufficient subjects for the study, if necessary, the age may be gradually increased to 70 years or decreased to 40 years
Healthy skin in the test areas.
Visible wrinkles in the face (grade 3 to 6 according to proderm wrinkle score) see appendix 2

### For Biopsy Subpanel:

Vaccination of tetanus within the last 10 years

### Exclusion Criteria

Female subjects: Pregnancy or lactation
Drug addicts, alcoholics
AIDS, HIV-positive or infectious hepatitis
Conditions which exclude a participation or might influence the test reaction/evaluation
Participation or being in the waiting period after participation in cosmetic and/or pharmaceutical studies pertaining to the test area
Active skin disease at the test area
Documented allergies to face/eye care products and food supplements or their ingredients
Intake of dietary supplements within the last 3 months before the start of the study
Diabetes mellitus
Cancer not being diagnosed as cured and requiring chemotherapy, irradiation and/or hormonal treatment within the last 2 years
One of the following illnesses with reduced physical capability/fitness: asthma (symptom-free allergic asthma is not an exclusion criterion), hypertension (if not adjusted with medication), cardiovascular diseases
Epilepsy
Wounds, moles, tattoos, scars, irritated skin, excessive hair growth, freckles, etc. at the test area that could influence the investigation
Regular use of tanning beds
Any topical medication at the test area within the last 3 days prior to the start of the study
Systemic therapy with immuno-suppressive drugs (e.g. corticosteroids) and/or antihistamines (e.g. antiallergics) and/or within the last 7 days prior to the start of the study
Therapy with antibiotics within the last 2 weeks prior to the start of the study
Past cosmetic surgery procedure in the test area (e.g. laser, facelift)
Cosmetic surgery procedure in the test area, e.g. IPL (Intensed Pulsed Light), botox, chemical peel, dermabrasion within the last 2 years prior to the start of the study and/or throughout the entire course of the study
Medical treatment for wrinkle reduction (e.g. peeling with vitamin A or fruit acids) on the face within the last 2 weeks prior to the start of the study

### For Biopsy Subpanel:

Regular medication with anti-coagulating drugs like Aspirin®, Macumar®, etc. (e.g. for thrombosis prophylaxis) within up to 15 days prior to the taking of the biopsies
History of complications at wound healing (e.g. keloids, hypertrophic scars or contracture scar)
Known intolerance to local anaesthetics
Known Sensitivity to any dressing systems

The aim of this proof-of-concept study is to investigate the impact on skin aging of a combination of a topical and oral product in comparison to the respective group receiving an oral placebo combined with the topical product. The topical product is a cosmetic designed to nourish the skin from the outside and the oral product is a food supplement improving skin health from within. The cosmetic product contains an active on skin aging and is acting on biological hallmarks of skin aging. The food supplement also contains several key bioactives designed to target different hallmarks of skin aging systemically.

Key biological pathways investigated in forearm skin biopsies include inflammatory and immune pathways, such as NF-κB, JAK/STAT, and interferon-regulated genes, which are often upregulated in autoimmune diseases, like lupus erythematosus and psoriasis. In aging or photoaging studies, oxidative stress and mitochondrial pathways, especially the Nrf2-antioxidant response and mitochondrial dysfunction signalling, are commonly assessed due to their role in cellular senescence and extracellular matrix breakdown. Moreover, analysis of epidermal barrier function pathways, such as those involving filaggrin, loricrin, and ceramide metabolism, is essential in conditions like atopic dermatitis. Modern systems biology approaches such as RNA sequencing enable unbiased pathway analysis, identifying both canonical and novel pathways implicated in skin pathology.

Skin biopsy samples will be analysed to study the activity of specific genes that are already known to play a role in important biological pathways related to skin and hair, such as inflammation, immune response, skin barrier function, and hair follicle biology. To support and guide our analysis, well-established, publicly available gene expression datasets that provide data from human skin, including from the forearm and scalp will be referenced. These datasets help to confirm the normal or altered expression patterns of key genes in the pathways we are studying. Examples of such datasets include:
GSE181549—A collection of gene expression data from forearm skin biopsies in systemic sclerosis and healthy individuals (NCBI GEO link: https://www.ncbi.nlm.nih.gov/geo/query/acc.cgi?acc=GSE181549).
GSE130955—Focused on known immune and fibrosis-related gene pathways in early systemic sclerosis (NCBI GEO link: https://www.ncbi.nlm.nih.gov/geo/query/acc.cgi?acc=GSE130955)
Human Protein Atlas—Skin tissue expression (proteinatlas.org)
Expression Atlas (EMBL-EBI)—For reviewing gene expression in healthy skin and in various dermatological conditions (Expression Atlas link: https://www.ebi.ac.uk/gxa/home)

These datasets provide validated references that support the interpretation of well-defined gene pathways in skin biology.

For this study female subjects aged preferably between 45 and 65 years with visible wrinkles in the face will be enrolled and divided into two treatment groups. One group will be assigned to the treatment group testing the active food supplement in combination with the active and placebo cosmetic test product in split-face design. The other group will be assigned to the treatment group testing the placebo food supplement in the same combination with the active and placebo formulations of the cosmetic test product in split-face design (see FIG. 2).

### Example 3

### Title of Clinical Study: a Randomized, Double-Blind, Placebo-Controlled Study to Evaluate the Safety and Efficacy of a Novel Supplement Comprising Urolithin A and Biotin in Males with Self-Perceived Thinning Hair

### the Following Criteria are Given for the Clinical Study:

### Inclusion Criteria:

Males between 35-60 years of age with self-perceived thinning
Voluntarily sign and date an informed consent agreement and photo release form approved by the Institutional Review Board.
Clinically confirmed to have hair thinning or loss by the investigator or qualified sub-investigator via physical exam, including only subjects with male pattern hair loss with frontal and/or vertex patterns II, IIA, III, IIIv and IV using the Norwood classification of patterned hair loss in males.
General good health, as determined by the Investigator or qualified sub-investigator

### Exclusion Criteria:

Clinical diagnosis of hair loss disorder such as alopecia areata, or scarring forms of alopecia.
Scalp hair loss on the treatment area, due to disease, injury, or medical therapy
Current skin disease (e.g., psoriasis, atopic dermatitis), in the opinion of the Investigator or qualified sub-investigator, might put the subject at risk with the study conduct or evaluations.
History of surgical correction of hair loss on the scalp (i.e. hair transplant).
Use of any products or devices purported to promote scalp hair growth (e.g., minoxidil) within the 6 months prior to the Baseline Visit.

Sample Size: n=100 subjects

Duration of Intervention: 6-months with visits at baseline, 3-months and end of study 6-months.

The compositions tested were compositions 1a to 11 of Example 1.

Where the supplement is taken orally, it comprises urolithin A between about 250 mg to about 145 g by weight.

Where the supplement is taken topically, it comprises urolithin A in the range of about 0.5% to about 2% by weight.

Optionally, the supplement comprises biotin in the range of 1 to 5 mg by weight and/or astaxanthin in the range of about 1 mg to about 30 mg by weight.

### Endpoints:

Changes in terminal hair counts as measured by phototrichogram
Changes in hair density as measured by phototrrichogram
Change in numbers of hairs shedded in Hair Pull Test
Subject self assessments of improvement in hair thickness and quality

To further evaluate the performance of our active ingredients, we have chosen to explore their effects on several protein and gene markers classically involved in hair growth or anti-greying processes.

The following markers are particularly important for hair growth:
Proliferation markers KI67 or PCNA
Hyperproliferation and differentiation markers K6/K16
Stem cell differentiation markers of the bulbar region such as Keration K15
Terminal differentiation markers Involucrine, filaggrin
Stem cell markers SOX9, LGR5, CD34, K19
Growth factors or diffusible factors such as:
VEGF involved in angiogenesis, essential for anagen growth,
IGF1 to stimulate follicular keratinocyte proliferation
KGF7 or KGF10 to stimulate follicle growth in the anagen phase
TGFb to control the catagen phase
BMP family proteins (2/4) to regulate stem cell differentiation
Wnt/β-catenin pathway crucial for stem cell activation and anagen induction

### for Anti-Greying:

To assess the anti-greying performance of our active ingredient, we explore:
Its ability to preserve melanocytes in the bulb of the hair follicle
Its ability to stimulate melanogenesis (melanin production) and regulate key markers such as TYR, TYRP
Its ability to protect against oxidative stress (major cause of pigment loss)
Its ability to maintain or regenerate melanocyte stem cells (in the bulge). Markers that are targeted are SOX10, or PAX3,

### Example 4. Synergistic Effects on Urolithin A and Astaxanthin on Mitochondrial Respiration

### Methods

Fatty acid oxidation analysis on C2C12 myotubes was measured with a Seahorse XF96 extracellular flux analyzer. C2C12s were seeded at 8,000 cells/well in an XF96 cell culture microplate. Once cells reached confluence, they were differentiated with DMEM (Gibco, 31966047) supplemented with 2% Horse serum, 1% PenStrep and 1% Hepes for 5 days. Cells were treated with UA 50 uM (Amazentis) and Astaxanthin 20 uM (MedChemExpress, LKT-A7476) for 24 hours in substrate-limited media (Gibco, A1443001) supplemented with 0.5 mM glucose, 1 mM glutamine, 1% Oleic acid and 0.5 mM L-Carnitine. Maximal respiration was monitored by injection of 3 UM FCCP in the cartridge. Prior to the assay, cells were incubated in a Fatty acid oxidation medium (111 mM NaCl, 4.7 mM KCl, 1.25 mM CaCI2, 2 mM MgSO4, 1.2 mM NaH2PO4, 5 mM L-Carnitine, 5 mM Hepes, 2.5 mM Glucose) for 20 min, solution must be adjusted to pH 7.4 at 37° C.

### Results

Mouse muscle C2C12 myotubes were treated for 24 hours with either Urolithin A alone, Astaxanthin alone or a combination of both compounds. Urolithin A alone induced mitochondrial respiration (+8.5%, Table 4). Astaxanthin alone induced mitochondrial respiration (+3.2% Table.4).

The combination of both Urolithin A and Astaxanthin showed a synergistic effect on mitochondrial respiration (+17.8%, Table 4).

TABLE 3
Table 3 indicates change in maximal mitochondrial respiration expressed
as % change for the indicated comparison. Corresponding statistical
significance is provided as adjusted p. value, calculated as One-Way
ANOVA.

| | % increase in basal | |
|---|---|---|
| | mitochondrial | Adjusted P |
| One Way ANOVA | respiration | Value |
| DMSO vs. UA 50 uM | +8.5% | 0.067 |
| DMSO vs. Astaxanthin 20 uM | +3.3% | 0.64 |
| DMSO vs. UA 50 uM + | +17.8% | <0.001 |
| Astaxanthin 20 uM | | |

### DMSO is Dimethyl Sulfoxide

The results are shown in Table 4 below where it can be seen that the combination of Astaxanthin and Urolithin A is far superior to the active ingredients alone . . .

TABLE 4

| | DMSO | UA | Astaxanthin | Astaxanthin 20 uM + |
|---|---|---|---|---|
| | 0.1% | 50 uM | 20 uM | UA 50 uM |
| Relative | 0.0% | 8.4% | 2.6% | 16.2% |
| Change (%) | | | | |
| Compared to | | | | |
| DMSO 0.1% | | | | |

### Example 5. Synergistic Anti-Inflammatory Effects of Urolithin A and Zinc in Normal Human Dermal Fibroblasts (nHDF)

The protective effects of urolithin A and Zinc against inflammation induced by a cytokines mix (Cytomix, CTX) in human dermal fibroblasts (nHDF cells) were investigated. A dose of 0.025 ng/ml of Cytokines mix was applied to induce inflammation, measured by quantifying the levels of the pro-inflammatory cytokine interleukin 6 (IL-6) in the cell culture supernatant. For the experiment, cells were first pre-treated with either i) urolithin A 2.5 uM, ii) Zinc at 100 uM, iii) a combination of urolithin A 2.5 uM and Zinc 100 uM, iv) or DMSO as control for 24 h. Then cells were co-treated with both CTX and either urolithin A or Zinc alone or in combination, as above. After 24h, IL-6 was measured in the cell supernatant to assess the ability of Urolithin A and Zinc to reduce Cytokines-induced inflammation.

Results indicate that CTX drastically increases IL-6 levels. Both urolithin A and Zinc treatment reduce CTX-mediated increase of IL-6 by 25% and 29.6% respectively (Table 5). The combination of both Urolithin A and Zinc resulted in a further synergistic decrease of IL-6 levels by 43.7%, compared to the treatment with the compounds alone (Table 5 and Table 6 below).

TABLE 5

| | | | | Combo |
|---|---|---|---|---|
| | CTX | UA | Zinc100 | Zinc 100 uM/ |
| | 0.025 ng/ml | 2.5 uM | uM | 2.5 uM UA |
| Relative | 0.00% | −25.05% | −29.57% | −43.69% |
| Change (%) | | | | |
| Compared | | | | |
| to CTX | | | | |

TABLE 6
Table 6 indicates the change in Interleukin-6 (IL-6) concentration in the
cell supernatant expressed as % changed for the indicated comparisons.
Corresponding statistical significance is provided as adjusted p. value,
calculated as One-Way ANOVA.

| | % reduction | Adjusted P |
|---|---|---|
| Ordinary One-Way ANOVA | IL-6 | Value |
| CTX vs. CTX + UA 2.5 uM | −25% | <0.023 |
| CTX vs. CTX + Zinc 100 uM | −29.6% | <0.0077 |
| CTX vs. CTX + UA 2.5 uM + Zinc 100 uM | −43.7% | <0.0002 |

### Methods

Normal Human dermal fibroblasts (nHDF) purchased from Promocell (ref. C-12302) were seeded at 6000 cells per well in a 96 well plate (Greiner) using a DMEM Glutamax 1 g D-glucose (Gibco, ref 21885108) supplemented with FBS 10% (PanBiotech, ref P30-3033), 1% PenStrep (Biowest) and 1% Hepes (Biowest). All media were sterilized with a 0.45 μm filter unit (VWR). The day after the seeded cells were treated for 24 h with 1) Urolithin A (UA) resuspended in DMSO, Zinc gluconate (MedChemExpress Ref. HY-W145499) or a combination of the two at the indicated doses. Cytokine mix (“cytomix”) was added in addition of the previous treatment for another 24h, cytokines were used equally for a total and final concentration of 0.025 ng/ml (mIFN-g (roche ref.11276905001), TNF-α (Peprotech ref. 300-01A) and IL-b (Proteintech ref. 200-01B)). The medium was collected and an ELISA for Interleukin-6 were performed following the instruction provided by the supplier (Proteintech ref. KE10007). Finally, the absorbance at a wavelength of 450 nm was measured using a microplate plate reader (Fluostar Optima, BMG Labtech). IL-6 data were finally normalized over cell number, calculated with a cell viability assay (Promega, ref. G7571).

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A composition comprising:
(a) a compound of formula (I), or a salt, prodrug, metabolite or derivative thereof; 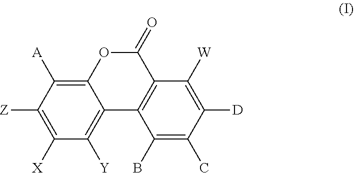
wherein:
A, B, C, D, W, X, Y and Z are each independently selected from H and OH; and
(b) one or more agents selected from agents for improving skin health, wherein the agents for improving skin health are selected from one or more of the following:
(i) one or more ceramides;
(ii) a carotenoid;
(iii) zinc or a salt thereof;
(iv) astaxanthin;
(v) L-lysine;
(vi) one or more NAD boosters;
(vii) eggshell membrane or an extract thereof; and/or
(viii) biotin, or a derivative thereof.

2. The composition as claimed in claim 1, comprising:
(a) a compound of formula (I), or a salt, prodrug, metabolite or derivative thereof; and
(b) one or more ceramides.

3. The composition as claimed in claim 2, further comprising one or more carotenoids.

4. The composition as claimed in claim 3, further comprising a zinc salt, for example, a zinc salt selected from zinc oxide, zinc sulphate, zinc acetate, zinc citrate, zinc gluconate, zinc picolinate, zinc orotate, and zinc bisglycinate.

5. The composition as claimed in claim 1, comprising
(a) a compound of formula (I), or a salt, prodrug, metabolite or derivative thereof;
(b) one or more phytoceramides;
(c) a carotenoid; and
(d) a zinc salt, for example, a zinc salt selected from zinc oxide, zinc sulphate, zinc acetate, zinc citrate, zinc gluconate, zinc picolinate, zinc orotate, and zinc bisglycinate.

6. The composition as claimed in claim 4, wherein the carotenoid is lycopene.

7. The composition as claimed in claim 4, further comprising (e) one or more NAD boosters.

8. The composition as claimed in claim 7, further comprising one or more agents selected from:
(f) eggshell membrane or an extract thereof;
(g) collagen;
(h) L-lysine;
(i) nicotinamide and/or reservatrol; and
(j) Miliacin encapsulated by polar lipid.

9. The composition as claimed in claim 1, comprising:
(a) a compound of formula (I), or a salt, prodrug, metabolite or derivative thereof; and
(b) biotin or a derivative thereof.

10. The composition as claimed in claim 9, wherein the biotin, or derivative thereof, is selected from free biotin, biocytin (e-biotin-L-Lysine), L-biotin sulphoxide, and D-biotin sulphoxide.

11. (canceled)

12. The composition as claimed in claim 1, wherein the compound of formula (I) is selected from urolithin A, urolithin B, urolithin C and urolithin D.

13. A composition comprising:
(a) a compound of formula (I), or a salt, prodrug, metabolite or derivative thereof; 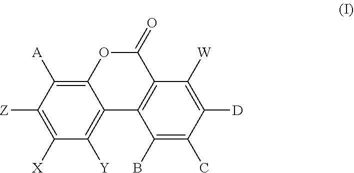
wherein:
A, B, C, D, W, X, Y and Z are each independently selected from H and OH; and
(b) an agent for improving skin health, wherein the agent for improving skin health is selected from zinc or a salt thereof and astaxanthin.

14. (canceled)

15. The composition as claimed in claim 13, further comprising:
(i) one or more ceramides;
(ii) a carotenoid;
(iii) L-lysine;
(iv) one or more NAD boosters;
(v) eggshell membrane or an extract thereof; and/or
(vi) biotin, or a derivative thereof.

16. A method of preventing or treating a skin condition, disease or disorder, comprising administering to a subject in need thereof a composition of claim 1, optionally wherein the skin condition, disease or disorder is a skin condition, disease or disorder associated with inadequate mitochondrial activity.

17. (canceled)

18. (canceled)

19. The method of claim 16, wherein the preventing or treating comprises:
a. improving skin health;
b. for maintaining or enhancing skin energy;
c. for maintaining or enhancing skin collagen;
d. reducing skin biological aging;
e. supporting healthy skin aging;
f. reducing skin wrinkles, and/or fine lines;
g. improving skin mitochondrial function;
h. increasing skin collagen levels;
i. improving skin hydration;
j. promoting replenishment of skin ceramide levels;
k. improving skin elasticity and barrier function;
l. protecting against free radical damage;
m. improving skin dryness;
n. improving photodamaged skin, and/or
o. Improving skin health in skin prone to redness, inflammation, acne or dryness.

20. The method of claim 16, further comprising administering a topical skin formulation comprising urolithin A to prevent or improve skin aging.

21. The composition as claimed in claim 20, wherein the topical skin formulation is a cosmetic topical cream comprising about 0.5 to about 5% of urolithin A.

22. The composition as claimed in claim 21, wherein the topical skin formulation comprises about 1% of urolithin A.

23. A cosmetic composition comprising a composition as claimed in claim 1.

24. A kit comprising:
(a) a composition as claimed in claim 1;
(b) a container, or containers, for containing said composition;
(c) optionally a topical skin formulation, comprising a compound of formula (I); and
(d) optionally instructions for simultaneous, separate or sequential administration with a topical skin formulation, comprising a compound of formula (I).

25. (canceled)
